SEQUENCE LISTING (27 sequences)

SEQ ID NO: 1 - protein, 125 aa, Artificial Sequence
  First extracellular Ig-like domain of SIRPalpha with mutation
EEELQVIQPDKSVSVAAGESAILHCTVTSLIPVGPIQWFRGAGPARELIYNQKEGHFPRVTTVSESTKRENMDFSISISAITPADAGTYYCVKFRKGSPDTEFKSGAGTELSVRAKPSAPVVSGP

SEQ ID NO: 2 - protein, 120 aa, Artificial Sequence [sequence omitted]

SEQ ID NO: 3 - protein, 107 aa, Artificial Sequence [sequence omitted]

SEQ ID NO: 4 - protein, 8 aa, Artificial Sequence
  HV-CDR-1 of IMM56
GYTFTDYW

SEQ ID NO: 5 - protein, 8 aa, Artificial Sequence
  HV-CDR-2 of IMM56
IYPGDGDT

SEQ ID NO: 6 - protein, 13 aa, Artificial Sequence
  HV-CDR-3 of IMM56
ARGDYYGSNSLDY

SEQ ID NO: 7 - protein, 6 aa, Artificial Sequence
  LV-CDR-1 of IMM56
QDVSTV

SEQ ID NO: 8 - protein, 3 aa, Artificial Sequence
  LV-CDR-2 of IMM56
SAS

SEQ ID NO: 9 - protein, 9 aa, Artificial Sequence
  LV-CDR-3 of IMM56
QQHYSPPYT

SEQ ID NO: 10 - protein, 330 aa, Artificial Sequence
  Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 107 aa, Artificial Sequence [sequence omitted]

SEQ ID NO: 12 - protein, 15 aa, Artificial Sequence
  Linker
GGGGSGGGGSGGGGS

SEQ ID NO: 13 - protein, 10 aa, Artificial Sequence
  linker
GGGGSGGGGS

SEQ ID NO: 14 - protein, 20 aa, Artificial Sequence
  Linker
GGGGSGGGGSGGGGSGGGGS

SEQ ID NO: 15 - protein, 590 aa, Artificial Sequence
  IMM5601's long chain (SIRPalphaD1-linker- IMM56's heavy chain)
EEELQVIQPDKSVSVAAGESAILHCTVTSLIPVGPIQWFRGAGPARELIYNQKEGHFPRVTTVSESTKRENMDFSISISAITPADAGTYYCVKFRKGSPDTEFKSGAGTELSVRAKPSAPVVSGPGGGGSGGGGSGGGGSQVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 1830 bp, Artificial Sequence
  IMM5601's long chain (SIRPalphaD1-linker- IMM56's heavy chain)
atgggatggtcatgtatcatcctttttctggtagcaactgcaactggagtacattcagaggaggagctgcaggtgattcagcctgacaagtccgtatcagttgcagctggagagtcggccattctgcactgcactgtgacctccctgatccctgtggggcccatccagtggttcagaggagctggaccagcccgggaattaatctacaatcaaaaagaaggccacttcccccgggtaacaactgtttcagagtccacaaagagagaaaacatggacttttccatcagcatcagtgccatcaccccagcagatgccggcacctactactgtgtgaagttccggaaagggagccctgacacggagtttaagtctggagcaggcactgagctgtctgtgcgtgccaaaccctctgcccccgtggtatcgggccctggcggcggtgggagcggcggcggtgggagcggcggcgggggctcgcaagtgcagctggtgcagagcggcgccgaggtggccaagcctggcacctccgtcaagctgagctgcaaggcctccggctacaccttcaccgactactggatgcagtgggtgaagcagagacctggccaaggcctggagtggatcggcaccatctaccctggcgacggcgacaccggctacgctcagaagttccaaggcaaggccaccctgaccgccgacaagagcagcaagaccgtgtacatgcacctgagcagcctggcctccgaggacagcgccgtgtactactgcgctagaggcgactactacggcagcaacagcctggactactggggccaaggcacaagcgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacgccacgtaccgtgtggtcagcgtcctcaccgtcctgcaccaagactggctgaatggcaaggagtacaagtgcaaggtctccaacaaagccctcccagcccccatcgccgcaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaatga

SEQ ID NO: 17 - protein, 214 aa, Artificial Sequence
  IMM5601's short chain (IMM56's light chain)
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - DNA, 841 bp, Artificial Sequence
  IMM5601's short chain (IMM56's light chain)
atgggatggtcatgtatcatcctttttctggtagcaactgcaactggagtacattcagacatcgtgatgacacagagccacctgagcatgagcacaagcctgggcgaccctgtgagcatcacctgcaaggcctcccaagacgtgagcaccgtggtggcctggtatcagcagaagcctggacagagccctagaagactgatctacagcgcctcctacagatacatcggcgtgcctgacagattcaccggcagcggcgccggcaccgacttcaccttcaccatcagcagcgtgcaagccgaggacctggccgtgtactactgtcagcagcactacagccctccttacaccttcggcggaggcaccaagctggagatcaagcgtgagttctagaggatccatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 19 - protein, 450 aa, Artificial Sequence
  IMM5602's long chain (IMM56's heavy chain)
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 1410 bp, Artificial Sequence
  IMM5602's long chain (IMM56's heavy chain)
atgggatggtcatgtatcatcctttttctggtagcaactgcaactggagtacattcacaagtgcagctggtgcagagcggcgccgaggtggccaagcctggcacctccgtcaagctgagctgcaaggcctccggctacaccttcaccgactactggatgcagtgggtgaagcagagacctggccaaggcctggagtggatcggcaccatctaccctggcgacggcgacaccggctacgctcagaagttccaaggcaaggccaccctgaccgccgacaagagcagcaagaccgtgtacatgcacctgagcagcctggcctccgaggacagcgccgtgtactactgcgctagaggcgactactacggcagcaacagcctggactactggggccaaggcacaagcgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacgccacgtaccgtgtggtcagcgtcctcaccgtcctgcaccaagactggctgaatggcaaggagtacaagtgcaaggtctccaacaaagccctcccagcccccatcgccgcaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaatga

SEQ ID NO: 21 - protein, 354 aa, Artificial Sequence
  IMM5602's short chain (SIRPalphaD1-linker- IMM56's light chain)
EEELQVIQPDKSVSVAAGESAILHCTVTSLIPVGPIQWFRGAGPARELIYNQKEGHFPRVTTVSESTKRENMDFSISISAITPADAGTYYCVKFRKGSPDTEFKSGAGTELSVRAKPSAPVVSGPGGGGSGGGGSGGGGSDIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - DNA, 1261 bp, Artificial Sequence
  IMM5602's short chain (SIRPalphaD1-linker- IMM56's light chain)
atgggatggtcatgtatcatcctttttctggtagcaactgcaactggagtacattcagaggaggagctgcaggtgattcagcctgacaagtccgtatcagttgcagctggagagtcggccattctgcactgcactgtgacctccctgatccctgtggggcccatccagtggttcagaggagctggaccagcccgggaattaatctacaatcaaaaagaaggccacttcccccgggtaacaactgtttcagagtccacaaagagagaaaacatggacttttccatcagcatcagtgccatcaccccagcagatgccggcacctactactgtgtgaagttccggaaagggagccctgacacggagtttaagtctggagcaggcactgagctgtctgtgcgtgccaaaccctctgcccccgtggtatcgggccctggcggcggtgggagcggcggcggtgggagcggcggcgggggctcggacatcgtgatgacacagagccacctgagcatgagcacaagcctgggcgaccctgtgagcatcacctgcaaggcctcccaagacgtgagcaccgtggtggcctggtatcagcagaagcctggacagagccctagaagactgatctacagcgcctcctacagatacatcggcgtgcctgacagattcaccggcagcggcgccggcaccgacttcaccttcaccatcagcagcgtgcaagccgaggacctggccgtgtactactgtcagcagcactacagccctccttacaccttcggcggaggcaccaagctggagatcaagcgtgagttctagaggatccatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 23 - protein, 364 aa, Artificial Sequence
  SIRPalphaD1 mutant-Fc (IMM01)
EEELQVIQPDKSVSVAAGESAILHCTVTSLIPVGPIQWFRGAGPARELIYNQKEGHFPRVTTVSESTKRENMDFSISISAITPADAGTYYCVKFRKGSPDTEFKSGAGTELSVRAKPSAPVVSGPAARATPQHEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - DNA, 1095 bp, Artificial Sequence
  SIRPalphaD1 mutant-Fc (IMM01)
gaggaggagctgcaggtgattcagcctgacaagtccgtatcagttgcagctggagagtcggccattctgcactgcactgtgacctccctgatccctgtggggcccatccagtggttcagaggagctggaccagcccgggaattaatctacaatcaaaaagaaggccacttcccccgggtaacaactgtttcagagtccacaaagagagaaaacatggacttttccatcagcatcagtgccatcaccccagcagatgccggcacctactactgtgtgaagttccggaaagggagccctgacacggagtttaagtctggagcaggcactgagctgtctgtgcgtgccaaaccctctgcccccgtggtatcgggccctgcggcgagggccacacctcagcacgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttga

SEQ ID NO: 25 - DNA, 57 bp, Artificial Sequence
  Murine IgG1 heavy chain signal peptide
atgggatggtcatgtatcatcctttttctggtagcaactgcaactggagtacattca

SEQ ID NO: 26 - DNA, 9 bp, Artificial Sequence
  Kozak
gccgccacc

SEQ ID NO: 27 - protein, 597 aa, Artificial Sequence
  Human SIRPalpha-murine IgG1 Fc
MGWSCIILFLVATATGVHSSCAWSGVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNEFVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

(19) United States
(12) Patent Application Publication
(10) Pub. No.: US 2023/0110702 A1
(43) Pub. Date: Apr. 13, 2023

(54) RECOMBINANT FUSION PROTEINS TARGETING CD47 AND CD38, PREPARATION AND USE THEREOF

(71) Applicant: ImmuneOnco Biopharmaceuticals (Shanghai) Co., Ltd., Shanghai (CN)

(72) Inventors: Wenzhi TIAN, Shanghai (CN); Song LI, Shanghai (CN); Dianze CHEN, Shanghai (CN)

(21) Appl. No.: 17/592,976

(22) Filed: Feb. 4, 2022

(30) Foreign Application Priority Data
Oct. 9, 2021 (CN) 202111179774.1

(51) Int. Cl. C07K 16/28; C07K 14/705; A61P 35/00

(52) U.S. Cl. CPC C07K 16/2896; C07K 14/70596; A61P 35/00; C07K 2319/30; C07K 2317/565; A61K 2039/505

(57) ABSTRACT

The present application provides a recombinant fusion protein containing an anti-CD38 antibody or an antibody fragment thereof, with at least one paratope of the anti-CD38 antibody or antibody fragment thereof linked via a linker to an extracellular Ig-like domain of a signal-regulatory protein (SIRP) at N-terminus of a heavy chain or a light chain constituting that paratope. The present application also provides a nucleic acid molecule encoding the recombinant fusion protein, an expression vector containing the nucleic acid molecule, a method for producing the recombinant fusion protein and a method for treating a disease associated with over expression of CD47 and/or CD38 using the recombinant fusion protein.

### RELATED APPLICATIONS AND INCORPORATION BY REFERENCE

This application claims priority to Chinese Patent Application No. 202111179774.1 filed on Oct. 9, 2021.

The foregoing application, and all documents cited therein or during its prosecution (“appln cited documents”) and all documents cited or referenced herein (including without limitation all literature documents, patents, published patent applications cited herein) (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Any Genbank sequences mentioned in this disclosure are incorporated by reference with the Genbank sequence to be that of the earliest effective filing date of this disclosure.

### SEQUENCE STATEMENT

The instant application contains a Sequence Listing which has been submitted electronically and is hereby incorporated by reference in its entirety. Said ASCII copy is named 55525_00053SL.txt and is 40.9 bytes in size.

### FIELD OF THE INVENTION

The application relates to a recombinant fusion protein targeting CD47, CD38 and/or FcR, and its preparation and use thereof, especially its use in tumor therapies.

### BACKGROUND OF THE INVENTION

Cancer cells have developed several mechanisms to evade hosts' immune surveillance, including: 1) to highly express CD38 proteins which may facilitate generation of extracellular adenosines whose immunosuppressive effects have been widely reported (Ohta A. et al., 2001); 2) to promote detachment of MICA/MICB from cancer cell membranes, which bind to NKG2D proteins on natural killer (NK) cell surfaces, blocking MICA/MICB⁺ cancer cell killing by NK cells; and 3) to express a high level of CD47s, which bind to the signal regulatory protein alpha (SIRPα) on macrophage surfaces, inducing inhibitory signals that inhibit phagocytosis of cancer cells by macrophages. It can be seen that the cancer cells are quite “smart” and reproduce quickly depending on their developed evasion mechanisms. Accordingly, development of effective anti-cancer drugs for killing the cancer cells may focus on targeting these mechanisms.

### SIRP and CD47

Signal regulatory protein (SIRP) is a trans-membrane glycoprotein, having three family members, SIRPα (CD172a), SIRPβ (CD172b) and SIRPγ (CD172g). The three proteins comprise similar extracellular regions but distinct intracellular domains. The extracellular region contains three immunoglobulin-like domains, one IgV-set and two IgC-set domains. The intracellular domain of SIRPα (CD172a) contains two inhibitory signaling regions that can inhibit signal transduction and corresponding cell functions. SIRPβ (CD172b) and SIRPγ (CD172g) have very short intracellular regions without any signal transduction domain. However, SIRPβ (CD172b) may function through an adaptor protein, e.g., DAP12 for signal transduction. SIRPs are mainly expressed on macrophages (Mφ), dendritic cells (DCs) and neurons.

CD47 is a transmembrane glycoprotein belonging to the immunoglobulin superfamily, and is expressed on the surface of all cell types including red blood cells. Ligands for CD47 include integrins, thrombospondin-1 and SIRPs. CD47, by interacting with SIRPα to emit a ‘don't eat me’ signal, can inhibit phagocytosis by macrophages and thus protects cells, such as blood cells, from being attacked by macrophages.

Studies have shown that many tumor or cancer cells over-express CD47s, which prevent their phagocytosis by macrophages. Such cancer cells include cells of acute myeloid leukemia (AML), chronic myeloid leukemia (CML), acute lymphoblastic leukemia (ALL), non-hodgkins lymphoma (NHL), multiple myeloma (MM), bladder cancer, ovarian cancer, prostate cancer, lung cancer, colon cancer, breast cancer, and pancreatic cancer. It is reported that injection of CD47 specific antibody that blocks the binding of CD47 to SIRPα can significantly inhibit tumor growth in tumor-bearing mice. Tumor or cancer cells were eliminated completely when the same antibody was injected into the mice carrying human leukemia cells (Theocharides APA, et al., 2012).

### CD38

CD38 is a glycoprotein found on the surfaces of many immune cells. It may function as a metabolic enzyme to catalyze the conversion of nicotinamide adenine dinucleotide (NAD⁺) to ADP ribose (ADPR) and cyclic ADP-ribose (cADPR), which two can regulate calcium signaling and modulate e.g., cell proliferation and T cell activation. Studies have shown CD38 is highly expressed on T cells, NK cell, and dendritic cells, and required for T cell priming, dendritic cell and neutrophil migration.

Many solid tumors, including liver cancer, non-small cell lung cancer, melanoma, pancreatic ductal adenocarcinoma, glioma, breast cancer, stomach cancer, and esophageal cancer, express CD38 at high levels (Dwivedi et al., 2021; Wo et al., 2020). For example, CD38⁺ tumor cells have been found in about 15-23% of lung cancer patients (Chen et al., 2018). CD38 may work with enzymes such as CD203 and CD73 to generate a large quantity of immunosuppressive adenosine. The extracellular adenosine may mediate recruitment of immunosuppressive cells such as Tregs and MDSCs so as to facilitate immunosuppression, and may also bind A2A receptors on e.g., NK cells, dendritic cells, and cytotoxic T cells to trigger immune cell exhaustion/dysfunction. Some hematological malignancies also highly express CD38s, and the CD38 levels were reported to be associated with poor prognosis in patients with e.g., multiple myeloma.

CD38 targeting antibodies revealed strong efficacy in treatment of hematological cancers. For example, daratumumab was approved to treat patients with relapsed and refractory multiple myeloma as a single therapy or in combination with other agents (Usmani et al., 2016). Further, daratumumab and isatuximab, another anti-CD38 antibody, are being tested in pre-clinical and clinical trials against multiple hematological and solid tumors, including plasma cell myeloma (e.g., multiple myeloma), lymphoma, pancreatic adenocarcinoma, non-small cell lung cancer, three-negative breast cancer, and prostate cancer (Dwivedi et al., 2021).

Daratumumab can bind CD38s expressed on tumor cells, and induce tumor cell death through mechanisms such as complement dependent cytotoxicity (CDC), antibody dependent cell mediated cytotoxicity (ADCC), and antibody dependent cellular phagocytosis (ADCP). The daratumumab therapy evidently reduced immunosuppressive cells with high CD38 expressions, such as Tregs and MDSCs, while significantly increasing cytotoxic CD8⁺ T cells. The triggered complement dependent cytotoxicity was reported to kill multiple myeloma cells even in the presence of immunosuppressive mesenchymal stromal cells isolated from bone marrow.

### Fc and FcR

The fragment crystallizable region (Fc region) is the tail region of an antibody and is the domain that determines the effector function of the antibody, that is, how it engages with specific cell receptors or other defense proteins.

An Fc receptor (FcR) is a protein found on the surface of certain cells, including B lymphocytes, follicular dendritic cells, natural killer cells, macrophages, neutrophils, eosinophils, basophils, and mast cells. These cells contribute to the protective functions of the immune system.

An Fc region may interact with Fc receptors and some proteins of the complement system, activating the immune system.

### Therapeutic Bi-Specific or Multi-Specific Fusion Proteins/Antibodies

Antibodies targeting a single tumor-associated antigen have been found to have limited therapeutic efficacy. For example, the overall response rate of an approved anti-PD-L1 antibody, avelumab (BAVENCIO®), is only 33%. And resistance to immunotherapies was observed in CD38-overexpressing tumors.

Bi- or tri-specific fusion proteins have been developed in recent years, showing promising effects in pre-clinical and clinical tests.

Although attaching additional binding moieties to conventional antibodies seems conceptually straightforward, such modification significantly alters antibody structures and may compromise one another's affinity and/or efficacy (Wang S et al., 2021). In order to optimize in vivo efficacy and pharmaceutical properties, elaborate design and engineering should be given to choice of main and appended binding moieties (sequences), balanced affinities for targets, sites of attachment (N- or C-termini, heavy or light chains), structural stability, linker lengths and/or sequences (Shim H. 2020).

U.S. Pat. No. 10,800,821 B2 discloses a recombinant bi-functional fusion protein of about 90 kDa, targeting both CD47 and FcR, which was used to treat Balb/c nude mice carrying HL cells, and an enhanced anti-tumor effect was observed.

Citation or identification of any document in this application is not an admission that such document is available as prior art to the present disclosure.

### SUMMARY OF THE INVENTION

The present application discloses a recombinant fusion protein, comprising an anti-CD38 antibody or an antibody fragment thereof, and a peptide specifically binding to CD47. Such a recombinant fusion protein has a comparable, if not higher, binding activity to CD38⁺ cells, CD47⁺ cells, and/or CD38⁺CD47⁺ cells, and induces comparable, if not higher, antibody dependent cell mediated cytotoxicity (ADCC) against CD38⁺CD47⁺ cells, compared to the monospecific anti-CD38 antibody and/or the CD47 binding peptide. The recombinant fusion protein of the disclosure even revealed better in vivo anti-tumor effects than the combination of the CD47 binding peptide and the anti-CD38 antibody.

Specifically, the present application discloses a recombinant fusion protein, comprising an anti-CD38 antibody or an antibody fragment thereof specifically binding CD38, and a CD47 binding peptide specifically binding to CD47, wherein the CD47 binding peptide is linked to the anti-CD38 antibody or antibody fragment thereof. The anti-CD38 antibody or antibody fragment thereof comprises a heavy chain variable region, a heavy chain constant region, and a light chain variable region, wherein the heavy chain variable region comprises a heavy chain variable region CDR1 (HV-CDR1), a heavy chain variable region CDR2 (HV-CDR2) and a heavy chain variable region CDR3 (HV-CDR3) having amino acid sequences set forth in SEQ ID NOs: 4, 5 and 6, respectively, the light chain variable region comprises a light chain variable region CDR1 (LV-CDR1), a light chain variable region CDR2 (LV-CDR2) and a light chain variable region CDR3 (LV-CDR3) having amino acid sequences set forth in SEQ ID NOs: 7, 8 and 9, respectively, and the heavy chain constant region has FcR binding affinity and is linked to C-terminus of the heavy chain variable region. The CD47 binding peptide comprises a mutated signal-regulatory protein (SIRP) extracellular domain, having an amino acid sequence having at least 95% sequence identity to SEQ ID NO: 1. The CD47 binding peptide may be linked to the N-terminus of the heavy chain variable region or the light chain variable region of the anti-CD38 antibody or antibody fragment thereof. The recombinant fusion protein of the disclosure can bind CD47, CD38 and FcR simultaneously.

The heavy chain variable region of the anti-CD38 antibody or antibody fragment thereof of the disclosure may comprise an amino acid sequence having at least 80%, 85%, 90%, 91%, 92%, 93%, 94%, 95%, 96%, 97%, 98%, 99% or 100% identity to SEQ ID NO: 2. The light chain variable region of the anti-CD38 antibody or antibody fragment thereof may comprise an amino acid sequence having at least 80%, 85%, 90%, 91%, 92%, 93%, 94%, 95%, 96%, 97%, 98%, 99% or 100% identity to SEQ ID NO: 3. In certain embodiments, the heavy chain variable region and the light chain variable region of the anti-CD38 antibody or antibody fragment thereof may comprise amino acid sequences having at least 80%, 85%, 90%, 91%, 92%, 93%, 94%, 95%, 96%, 97%, 98%, 99% or 100% identity to SEQ ID NOs: 2 and 3, respectively.

The heavy chain constant region having FcR binding affinity may be naturally occurring or artificially engineered human IgG1, IgG2, IgG3 or IgG4 heavy chain constant region, or a functional fragment thereof. In certain embodiments, the heavy chain constant region having FcR binding affinity is human IgG1 heavy chain constant region, or a functional fragment thereof. In certain embodiments, the heavy chain constant region having FcR binding affinity comprises the amino acid sequence of SEQ ID NO: 10.

The anti-CD38 antibody or antibody fragment thereof may comprise a light chain constant region, such as human kappa light chain constant region, or a functional fragment thereof, linked to the C-terminus of the light chain variable region. In certain embodiments, the anti-CD38 antibody or antibody fragment thereof may comprise the amino acid sequence of SEQ ID NO: 11.

The heavy chain of the anti-CD38 antibody or antibody fragment thereof of the disclosure may comprise an amino acid sequence having at least 80%, 85%, 90%, 91%, 92%, 93%, 94%, 95%, 96%, 97%, 98%, 99% or 100% identity to SEQ ID NO: 19. The light chain of the anti-CD38 antibody or antibody fragment thereof may comprise an amino acid sequence having at least 80%, 85%, 90%, 91%, 92%, 93%, 94%, 95%, 96%, 97%, 98%, 99% or 100% identity to SEQ ID NO: 17. In certain embodiments, the heavy chain and the light chain of the anti-CD38 antibody or antibody fragment thereof may comprise amino acid sequences having at least 80%, 85%, 90%, 91%, 92%, 93%, 94%, 95%, 96%, 97%, 98%, 99% or 100% identity to SEQ ID NOs: 19 and 17, respectively.

In certain embodiments, at least one paratope of the anti-CD38 antibody or antibody fragment thereof is linked to the CD47 binding peptide at N-terminus of the heavy chain variable region or the light chain variable region constituting the paratope. In certain embodiments, each paratope of the anti-CD38 antibody or antibody fragment thereof is linked to the CD47 binding peptide at N-terminus of the heavy chain variable region or the light chain variable region constituting the paratope. In certain embodiments, each paratope of the anti-CD38 antibody or antibody fragment thereof is linked to the CD47 binding peptide at N-terminus of the heavy chain variable region constituting the paratope. In certain embodiments, each paratope of the anti-CD38 antibody or antibody fragment thereof is linked to the CD47 binding peptide at N-terminus of the light chain variable region constituting the paratope.

The anti-CD38 antibody or antibody fragment thereof may be linked to the CD47 binding peptide via a linker. The linker may be a peptide linker made up of 5 to 30, 10 to 30, 10 to 20, or 15 amino acids. The linker may be e.g., -(Gly-Gly-Gly-Gly-Ser)₂-(SEQ ID NO: 13), -(Gly-Gly-Gly-Gly-Ser)₃-(SEQ ID NO: 12), or -(Gly-Gly-Gly-Gly-Ser)₄-(SEQ ID NO: 14). In certain embodiments, the linker is -(Gly-Gly-Gly-Gly-Ser)₃-(SEQ ID NO: 12).

The recombinant fusion protein of the disclosure may comprise a CD47 binding peptide-linker-anti-CD38 heavy chain fragment and an anti-CD38 light chain, wherein the CD47 binding peptide-linker-anti-CD38 heavy chain fragment may comprise an amino acid sequence having at least 80%, 85%, 90%, 91%, 92%, 93%, 94%, 95%, 96%, 97%, 98%, 99% or 100% identity to SEQ ID NO: 15, the anti-CD38 light chain may comprise an amino acid sequence having at least 80%, 85%, 90%, 91%, 92%, 93%, 94%, 95%, 96%, 97%, 98%, 99% or 100% identity to SEQ ID NO: 17. The recombinant fusion protein of the disclosure may comprise a CD47 binding peptide-linker-anti-CD38 heavy chain fragment and an anti-CD38 light chain variable region, wherein the CD47 binding peptide-linker-anti-CD38 heavy chain fragment may comprise an amino acid sequence having at least 80%, 85%, 90%, 91%, 92%, 93%, 94%, 95%, 96%, 97%, 98%, 99% or 100% identity to SEQ ID NO: 15, the anti-CD38 light chain variable region may comprise an amino acid sequence having at least 80%, 85%, 90%, 91%, 92%, 93%, 94%, 95%, 96%, 97%, 98%, 99% or 100% identity to SEQ ID NO: 3. The amino acid sequences of SEQ ID NOs: 15 and 17 may be encoded by nucleotide sequences of SEQ ID NOs: 16 and 18, respectively.

The recombinant fusion protein of the disclosure may comprise an anti-CD38 heavy chain, and a CD47 binding peptide-linker-anti-CD38 light chain fragment, wherein the anti-CD38 heavy chain may comprise an amino acid sequence having at least 80%, 85%, 90%, 91%, 92%, 93%, 94%, 95%, 96%, 97%, 98%, 99% or 100% identity to SEQ ID NO: 19, and the CD47 binding peptide-linker-anti-CD38 light chain fragment may comprise an amino acid sequence having at least 80%, 85%, 90%, 91%, 92%, 93%, 94%, 95%, 96%, 97%, 98%, 99% or 100% identity to SEQ ID NO: 21. The amino acid sequences of SEQ ID NOs: 19 and 21 may be encoded by the nucleotide sequences of SEQ ID NOs: 20 and 22, respectively.

The present application also provides a nucleic acid molecule encoding the recombinant fusion protein of the disclosure, as well as an expression vector comprising such a nucleic acid molecule and a host cell comprising such an expression vector. A method for preparing the recombinant fusion protein using the host cell of the disclosure is provided, comprising steps of (i) expressing the recombinant fusion protein in the host cell, and (ii) isolating the recombinant fusion protein from the host cell or its cell culture.

The application further provides a pharmaceutical composition which may comprise the recombinant fusion protein, the nucleic acid molecule, the expression vector, or the host cell, of the disclosure, and at least one pharmaceutically acceptable excipient. In certain embodiments, the pharmaceutical composition comprises at least one pharmaceutically acceptable adjuvant.

The recombinant fusion protein or pharmaceutical composition of the disclosure may be used in treatment of, or in preparation of a medicament for treating a disease associated with over-expression of CD47 and/or CD38.

In one aspect, the present application provides a method for treating or alleviating a disease associated with over-expression of CD47 and/or CD38 in a subject in need thereof, comprising administering to the subject a therapeutically effective amount of the pharmaceutical composition of the disclosure.

The disease associated with over-expression of CD47 and/or CD38 may be a hematological tumor, or a solid tumor. The disease associated with over-expression of CD47 and/or CD38 may include, but not limited to, acute myelocytic leukemia (AML), chronic myelocytic leukemia (CML), acute lymphoblastic leukemia (ALL), lymphoma, multiple myeloma (MM), bladder cancer, ovarian cancer, prostate cancer, lung cancer, colon cancer, breast cancer, pancreatic adenocarcinoma, melanoma, glioma, esophageal cancer, and plasma cell myeloma.

The disclosure further provides a method for enhancing immune responses, and/or restoring immune cell functions in a subject in need thereof, comprising administering to the subject a therapeutically effective amount of the pharmaceutical composition of the disclosure. In certain embodiments, the subject is human.

Other features and advantages of the instant disclosure will be apparent from the following detailed description and examples which should not be construed as limiting. The contents of all references, GenBank entries, patents and published patent applications cited throughout this application are expressly incorporated herein by reference.

Accordingly, it is an object of the application not to encompass within the application any previously known product, process of making the product, or method of using the product such that Applicants reserve the right and hereby disclose a disclaimer of any previously known product, process, or method. It is further noted that the application does not intend to encompass within the scope of the application any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the application to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the application.

### DESCRIPTION OF THE DRAWINGS

The following detailed description, given by way of example, but not intended to limit the application solely to the specific embodiments described, may best be understood in conjunction with the accompanying drawings.

FIG. 1 is the schematic diagram of structures of the recombinant fusion proteins, IMM5601 and IMM5602, of the present application. The circular “SIRPαD1” domain represents mutated extracellular domain 1 of SIRP alpha protein (SIRPαD1), which is linked via a linker to the N-terminus of heavy chains (left panel, IMM5601) or light chains (right panel, IMM5602) of an anti-CD38 antibody IMM56. The SIRPαD1 mutant and the linker comprise the amino acid sequences of SEQ ID NOs: 1 and 12, respectively. The IMM56′ heavy chain and light chain comprise the amino acid sequences of SEQ ID NOs: 19 and 17, respectively.

FIG. 2 shows the binding activity of IMM5601 and IMM5602 to CD38⁺CD47⁺ Raji cells, with IMM56 and IgG used as the positive and negative controls, respectively.

FIG. 3 shows the binding activity of IMM5601 and IMM5602 to CD38⁺CD47⁺ U266 cells, with IMM56 and IgG used as the positive and negative controls, respectively.

FIG. 4 shows the binding activity of IMM5601 to CD38⁺CD47⁺ NCI-H929 cells, with IMM56 and IMM01 used as positive controls, and IgG as the negative control. IMM01, disclosed in US2021/0024598A1, comprises two copies of SIRPαD1 mutant (SEQ ID NO: 1) linked to two Fc fragments, and the monomer in the formed dimer has the amino acid sequence of SEQ ID NO: 23.

FIG. 5 shows the binding activity of IMM5601 and IMM5602 to CD38⁻CD47⁺ Jurkat cells, with IMM01 used as the positive control, and IMM56 and IgG as negative controls.

FIG. 6 shows the capability of IMM5601 to block binding of SIRPα-Fc to CD38⁺CD47⁺ NCI-H929 cells, with IMM01 used as the positive control, IMM56 and IgG as the negative controls.

FIGS. 7A and 7B show the capability of IMM5601 and IMM5602 to induce antibody dependent cell mediated cytotoxicity against CD38⁺CD47⁺ Raji cells (A) and U266 cells (B), with IMM56 and IgG used as the positive and negative controls, respectively.

FIG. 8 shows the capability of IMM5601 to induce antibody dependent cell mediated cytotoxicity against CD38⁺CD47⁺ NCI-H929 cells, with IMM01 and IMM56 used as positive controls, and IgG as the negative control.

FIG. 9 shows the in vivo anti-tumor efficacy of IMM01, IMM56, IMM5601, and the IMM56+IMM01 combination in CB17-SCID mice injected with CD38⁺CD47⁺ NCI-H929 cells.

### DETAILED DESCRIPTION OF THE APPLICATION

There are principally three different approaches to targeting two or more pharmacological targets of tumor growth. Most commonly, patients can be given a cocktail of two or more different drugs. Although this option allows for maximal flexibility with respect to possible drug combinations and different dosages, it suffers from (a) potentially poor adherence to treatment by the patient because of the increased pill burden and the different dosing schedules for the individual drugs, (b) possible incompatibilities because of drug-drug interactions, and (c) increased risk of drug side effects. These problems can reduce the effectiveness of therapy and hamper the attainment of treatment goals particularly in the management of chronic diseases such as cancer.

The second approach relies on the use of fixed-dose combinations of drugs in a single dosage form. This approach reduces pill burden, resulting in improved patient compliance. The disadvantage of fixed-dose combinations is primarily the limited choice of possible dose ratios between the active ingredients, which makes it more difficult to properly titrate the individual patient to maximum efficacy with minimal adverse effects. In addition, different pharmacokinetic properties of the components in the combination might lead to a complex temporal mismatch in pharmacodynamic effects at the individual targets thereby compromising overall efficacy.

The third approach is the use of multifunctional drugs that combine two or more pharmacologies in a single compound. The design and validation of such multifunctional molecules are more complex and require substantial investigation into the optimal ratio of target activities in the molecule, but the unified pharmacokinetics may yield matched pharmacodynamic activities at the molecular targets. Multifunctional molecules may also be amenable to fixed dose combination with other drugs thereby combining three or even four pharmacologies in a single pill to produce further increments in efficacy.

Through diligent experimentation, the present inventors have invented a novel recombinant multi-functional fusion protein, which can attack tumors, via three mechanisms of actions, one to reduce the generation of extracellular adenosines, decreasing immunosuppressive cell recruitment, and releasing CD38 mediated immune cell exhaustion or dysfunction, one to release the check on macrophages by SIRP-mediated inhibitory signals, the third to stimulate cancer cell killings by NK cells and/or macrophages.

The recombinant fusion protein of the present application comprises an anti-CD38 antibody or an antibody fragment thereof, with at least one paratope of the antibody or antibody fragment linked via a linker to an extracellular Ig-like domain of a signal-regulatory protein (SIRP) at the N-terminus of a heavy chain variable region or a light chain variable region constituting the paratope. The recombinant protein can bind to CD47, CD38 and FcR simultaneously, i) reducing extracellular adenosine levels caused by CD38 overexpression on tumor cells, to decrease recruitment of immunosuppressive cells, and to release CD38 mediated immune cell exhaustion/dysfunction; ii) blocking the interaction of CD47s on cancer cells with SIRPs on macrophages and thus releasing the check on macrophages by SIRP-mediated inhibitory signals; and iii) binding Fc portion of the antibody to FcRs on NK cells and/or macrophages to stimulate cancer cell killings by NK cells and/or macrophages. In an embodiment, one paratope of the anti-CD38 antibody or antibody fragment thereof is linked via a linker to an extracellular Ig-like domain of a signal-regulatory protein (SIRP) at the N-terminus of a heavy chain variable region or a light chain variable region constituting the paratope. In another embodiment, each paratope of the anti-CD38 antibody or antibody fragment thereof is linked via a linker to an extracellular Ig-like domain of a signal-regulatory protein (SIRP) at the N-terminus of a heavy chain variable region or a light chain variable region constituting the paratope. In one embodiment, each paratope of the anti-CD38 antibody or antibody fragment thereof is linked via a linker to an extracellular Ig-like domain of a signal-regulatory protein (SIRP) at the N-terminus of a heavy chain variable region constituting the paratope. In one embodiment, each paratope of the anti-CD38 antibody or antibody fragment thereof is linked via a linker to an extracellular Ig-like domain of a signal-regulatory protein (SIRP) at the N-terminus of a light chain variable region constituting the paratope. The recombinant fusion protein of the present application is small in size (150-180 kDa) and has a long half-life of 5-10 days.

The three main components contained in the recombinant fusion protein of the present application are the extracellular Ig-like domain of a signal-regulatory protein (SIRP), the linker, and the anti-CD38 antibody. A person of ordinary skills in the art will recognize that there are many design choices for selecting the above three components. Preferably, human-derived sequence is used in human cancer therapies, as the strong immunogenicity of the proteins or peptides from non-human animals may lead to allergy and other adverse effects. However, other animal proteins or peptides, humanized if appropriate, may also be used in the present application based on different application purposes.

Any extracellular Ig-like domain of any SIPR (SIRPα, SIRPβ, and SIRPγ) capable of binding with CD47 may be selected for construction of the recombinant fusion protein. In one embodiment, the signal-regulatory protein in the recombinant fusion protein is SIRPα, and the extracellular Ig-like domain of the signal-regulatory protein is the first extracellular Ig-like domain of SIRPα (SIRPαD1). In certain embodiments, SIRPαD1 is a SIRPαD1 mutant that contains an N→A mutation at position 80 of SEQ ID NO: 1 to remove a glycosylation site.

In one embodiment, the recombinant fusion protein may comprise SIRPαD1 having the amino acid sequence set forth in SEQ ID NO: 1. In another embodiment, the SIRPαD1 may comprise an amino acid sequence having at least 80%, 85%, 90%, 91%, 92%, 93%, 94%, 95%, 96%, 97%, 98% or 99% sequence identity to SEQ ID NO: 1, wherein the SIRPαD1 can bind to CD47s on the cell surface of cancer/tumor cells and block the interaction of CD47s with SIRPs on the cell surfaces of macrophages.

Linkers serve primarily as a spacer between the extracellular Ig-like domain of SIRP and the N-terminus of the heavy chain or light chain of an anti-CD38 antibody. The linker may be made up of amino acids linked together by peptide bonds, preferably from 5 to 30 amino acids, from 10 to 30 amino acids, from 10 to 20 amino acids, or 15 amino acids, linked by peptide bonds, wherein the amino acids are selected from the 20 naturally occurring amino acids. One or more of these amino acids may be glycosylated or deglycosylated, as is understood by those of skill in the art. In one embodiment, the 5 to 30, 10 to 30, 10 to 20, or 15 amino acids may be selected from glycine, alanine, proline, asparagine, glutamine, serine and lysine. In one embodiment, a linker is made up of a majority of amino acids that are sterically unhindered, such as glycine and alanine. Exemplary linkers are polyglycines, particularly Glys, poly(Gly-Ala), and polyalanines. One exemplary suitable linker as shown in the Examples below is -(Gly-Ser)ₙ-, such as -(Gly-Gly-Gly-Gly-Ser)₃-(SEQ ID NO: 12).

Linkers may also be non-peptide linkers. For example, alkyl linkers such as —NH—, —(CH₂)s-C(O)—, wherein s=2-20 can be used. These alkyl linkers may further be substituted by any non-sterically hindering group such as lower alkyl (e.g., C14) lower acyl, halogen (e.g., CI, Br), CN, NH₂, phenyl, etc.

In certain embodiments, the anti-CD38 antibody may be an isolated monoclonal IgG antibody comprising two heavy chains and two light chains, wherein each heavy chain may comprise a heavy chain variable region and a heavy chain constant region, and each light chain may comprise a light chain variable region and optionally a light chain constant region. The heavy chain variable region and the light chain variable region of the anti-CD38 antibody may comprise amino acid sequences set forth in SEQ ID NOs: 2 and 3, respectively. The heavy chain constant region may comprise the amino acid sequence of SEQ ID NO: 10. The light chain constant region may comprise the amino acid sequence of SEQ ID NO: 11. The Fab portion (or paratope) of the anti-CD38 antibody may bind to CD38s on the cell surfaces of e.g., cancer/tumor cells to block the generation of extracellular adenosines, reducing immunosuppressive cell recruitment and immune cell exhaustion, while the Fc portion of the anti-CD38 antibody may bind to FcRs on the cell surfaces of NK cells or macrophages to stimulate cancer cell killings by the NK cells or macrophages. In certain embodiments, the heavy chain variable region may comprise an amino acid sequence having at least 80%, 85%, 90%, 91%, 92%, 93%, 94%, 95%, 96%, 97%, 98%, 99% or 100% sequence identity to SEQ ID NO: 2, and the heavy chain constant region may comprise an amino acid sequence having at least 80%, 85%, 90%, 91%, 92%, 93%, 94%, 95%, 96%, 97%, 98%, 99% or 100% sequence identity to SEQ ID NO: 10, wherein the anti-CD38 antibody is able to bind to CD38s on cancer/tumor cells and block the generation of extracellular adenosines, and is also able to bind to FcRs on NK cells or macrophages and thus activate the NK cells or macrophages to kill the cancer cells. In certain embodiments, the light chain variable region may comprise an amino acid sequence having at least 80%, 85%, 90%, 91%, 92%, 93%, 94%, 95%, 96%, 97%, 98%, 99% or 100% sequence identity to SEQ ID NO: 3, wherein the anti-CD38 antibody is able to bind to CD38s on cancer/tumor cells and block the generation of extracellular adenosines.

The term “antibody” as referred to herein includes whole antibodies of e.g., IgG, IgA, IgD, IgE and IgM, and any antigen binding fragment (i.e., “antigen-binding portion”) or single chains thereof. Whole antibodies are glycoproteins comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. A “functional fragment” of a heavy chain constant region refers to a part of the constant region that retains the capability to mediate binding of the immunoglobulin to host tissues or factors, including various cells of the immune system (e.g., effector cells) and the first component (C1q) of the classical complement system, to initiate e.g., ADCC, CDC, ADCP and the like.

The term “antibody fragment” herein refers to a portion or fragment of an anti-CD38 antibody of the disclosure that retains the ability to specifically bind to CD38s, and optionally the ability to bind Fc receptors.

The heavy chain variable region CDRs and the light chain variable region CDRs in the antibody or antibody fragment thereof of the disclosure have been defined by the IMGT numbering system. However, as is well known in the art, CDR regions can also be determined by other systems such as Chothia, Kabat, AbM, or Contact numbering system/method, based on heavy chain/light chain variable region sequences.

The term “antibody dependent cellular cytotoxicity”, “antibody dependent cell-mediated cytotoxicity” or “ADCC” refers to a mechanism of cell-mediated immune defense whereby an effector cell of the immune system actively lyses a target cell bound by e.g., the anti-CD38 antibodies and/or SIRPα-Fc molecules.

The term “subject” includes any human or nonhuman animal. The term “nonhuman animal” includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses.

As used herein, “sequence identity” refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using the publicly available computer software such as ClustalOmega, T-coffee, Kalign and MAFFT. When using such softwares, the default parameters, e.g. for gap penalty and extension penalty, are preferably used.

Also, the present application provides a polynucleotide molecule encoding the recombinant fusion protein and an expression vector expressing the recombinant fusion protein. Examples of vectors include but are not limited to plasmids, viral vectors, yeast artificial chromosomes (YACs), bacterial artificial chromosomes (BACs), transformation-competent artificial chromosomes (TACs), mammalian artificial chromosomes (MACs) and human artificial episomal chromosomes (HAECs).

The present application provides host cells comprising the above expression vectors. The host cells may be transformed or transfected with the expression vectors. Suitable host cells include Escherichia coli, yeasts and other eukaryotes. Preferably, Escherichia coli, yeast or mammalian cell lines (such as COS or CHO) are used.

In another aspect, the present disclosure provides a pharmaceutical composition comprising the recombinant fusion protein of the present application formulated together with a pharmaceutically acceptable excipient, or a pharmaceutically acceptable adjuvant. The composition may optionally contain one or more additional pharmaceutically active ingredients, such as another antibody or a drug. The pharmaceutical compositions of the application also can be administered in a combination therapy with, for example, another immune-stimulatory agent, anti-cancer agent, or a vaccine.

The pharmaceutical composition can comprise any number of excipients. Excipients that can be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients are taught in Gennaro, ed., Remington: The Science and Practice of Pharmacy, 20th Ed. (Lippincott Williams & Wilkins 2003), the disclosure of which is incorporated herein by reference.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in injection. For example, the vehicle or carrier may be neutral buffered saline or saline mixed with serum albumin Other exemplary pharmaceutical compositions comprise Tris buffers, or acetate buffers, which may further include sorbitol or a suitable substitute thereof. In one embodiment of the present application, compositions may be prepared for storage by mixing the selected composition having the desired degree of purity with optional formulation agents (Remington's Pharmaceutical Sciences, supra) in the form of a lyophilized cake or an aqueous solution. Further, the therapeutic composition may be formulated as a lyophilizate using appropriate excipients such as sucrose.

Preferably, the pharmaceutical composition is suitable for intravenous, intramuscular, subcutaneous, parenteral, spinal or epidermal administration (e.g., by injection or infusion). Depending on the route of administration, the active molecule can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the application can be administered via a non-parenteral route, such as a topical, epidermal or mucosal route of administration, e.g., intranasally, orally, vaginally, rectally, sublingually or topically.

Pharmaceutical compositions can be in the form of sterile aqueous solutions or dispersions. They can also be formulated in a microemulsion, liposome, or other ordered structure suitable to high drug concentration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01% to about 99% of active ingredient in combination with a pharmaceutically acceptable carrier.

Dosage regimens are adjusted to provide the optimum desired response (e.g., a therapeutic response). For example, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. Alternatively, the fusion protein can be administered as a sustained release formulation, in which case less frequent administration is required.

For administration of the fusion protein, the dosage ranges from about 0.0001 to 100 mg/kg of the host body weight. An exemplary treatment regime entails administration twice per week.

A “therapeutically effective dosage” of a recombinant fusion protein of the application preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective dosage” preferably inhibits tumor growth by at least about 40%, more preferably by at least about 60%, even more preferably by at least about 80%, and still more preferably by at least about 99% relative to untreated subjects. A therapeutically effective amount of a recombinant fusion protein of the present application can decrease tumor size, or otherwise ameliorate symptoms in a subject, which is typically a human or can be another mammal.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparatuses (U.S. Pat. Nos. 4,447,233 and 4,447,224); and (5) osmotic devices (U.S. Pat. Nos. 4,439,196 and 4,475,196); the disclosures of which are incorporated herein by reference.

In certain embodiments, the fusion protein of the application can be formulated to ensure proper distribution in vivo. For example, to ensure that the therapeutic fusion proteins of the application cross the blood-brain barrier, they can be formulated in liposomes, which may additionally comprise targeting moieties to enhance selective transport to specific cells or organs. See, e.g. U.S. Pat. Nos. 4,522,811; 5,374,548; 5,416,016; and 5,399,331.

A gene therapy in vivo is also envisioned wherein a nucleic acid molecule encoding the recombinant fusion protein of the present application, or a derivative thereof is introduced directly into the subject. For example, a nucleic acid sequence encoding a recombinant fusion protein of the present application is introduced into target cells via local injection of a nucleic acid construct with or without an appropriate delivery vector, such as an adeno-associated virus vector. Alternative viral vectors include, but are not limited to, retroviruses, adenovirus, herpes simplex vims and papilloma virus vectors. Physical transfer of the virus vector may be achieved in vivo by local injection of the desired nucleic acid construct or other appropriate delivery vector containing the desired nucleic acid sequence, liposome-mediated transfer, direct injection (naked DNA), or microparticle bombardment (gene-gun).

The compositions of the present disclosure may be used alone or in combination with other therapeutic agents to enhance their therapeutic effects or decrease potential side effects.

Another object of the present application is to provide a method for preparing the above recombinant fusion protein and the pharmaceutical composition comprising the same. In one embodiment, the method comprises (1) providing a protein-encoding polynucleotide molecule; (2) constructing an expression vector comprising the polynucleotide molecule of (1); (3) transfecting or transforming suitable host cells with the expression vector of (2) and cultivating the host cells to express the protein; and (4) purifying the protein. The preparation may be carried out with well-known technologies by an ordinarily skilled artisan.

Another object of the present application is to provide a method of treating cancer using the pharmaceutical composition of the present application, comprising administrating an effective amount of the aforementioned pharmaceutical composition to the patients or subjects in need thereof. In one embodiment, the pharmaceutical composition is used to treat CD47 and/or CD38 overexpressing tumors or cancers, including, but not limited to, acute myelocytic leukemia (AML), chronic myelocytic leukemia (CML), acute lymphoblastic leukemia (ALL), lymphoma, multiple myeloma (MM), bladder cancer, ovarian cancer, prostate cancer, lung cancer, colon cancer, breast cancer, pancreatic adenocarcinoma, melanoma, glioma, esophageal cancer, and plasma cell myeloma.

In one embodiment, the disease related to over-expressions of CD47 and/or CD38 includes, but not limited to, Crohn's disease, allergic asthma, and rheumatoid arthritis.

The pharmaceutical composition of the disclosure may also be used to enhance immune responses, and/or restore immune cell functions.

The present application is now further described with the non-limiting examples below.

### EXAMPLES

The structures of the exemplary recombinant proteins of the disclosure, IMM5601 and IMM5602, and control proteins, will be described below in further details.

IMM56 is an IgG antibody, comprising two heavy chains and two light chains, wherein the heavy chain variable region, the heavy chain constant region, the light chain variable region and the light chain constant region comprise the amino acid sequences of SEQ ID NOs: 2, 10, 3 and 11, respectively.

IMM5601 comprises IMM56 and two SIRPαD1s (SEQ ID NO: 1), each SIRPαD1 linked via a linker (SEQ ID NO: 12) to the N terminus of each heavy chain of IMM56. IMM56 comprises a heavy chain of SEQ ID NO: 19, and a light chain of SEQ ID NO: 17.

IMM5602 comprises IMM56 and two SIRPαD1s (SEQ ID NO: 1), each SIRPαD1 linked via a linker (SEQ ID NO: 12) to the N terminus of each light chain of IMM56. IMM56 comprises a heavy chain of SEQ ID NO: 19, and a light chain of SEQ ID NO: 17.

IMM01 was disclosed in US2021/0024598A1, comprising two SIRPαD1s (SEQ ID NO: 1) linked to two Fc fragments, wherein each monomer of the formed dimer has the amino acid sequence and nucleotide sequence of SEQ ID NOs: 23 and 24.

### Example 1. Vector Construction and Protein Expression

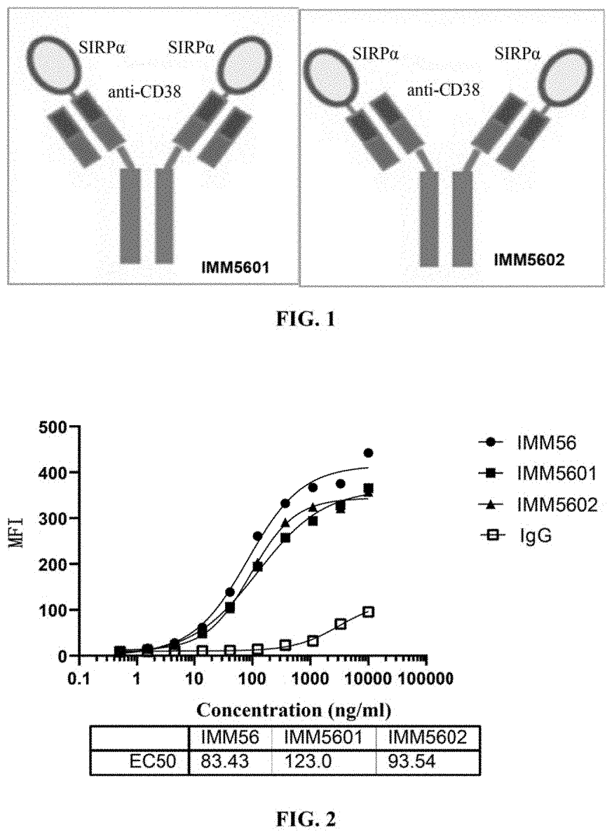

The structures of IMM5601 and IMM5602 were shown in FIG. 1, and the full length coding sequences of the recombinant fusion proteins were designed artificially.

Specifically, for IMM5601's long chain, i.e., the SIRPαD1-linker-anti-CD38 heavy chain fragment, 57 nucleotides encoding the signal peptide of mouse IgG1 heavy chain (SEQ ID NO: 25) were added to the 5′ end of the coding sequence of the SIRPαD1-linker-anti-CD38 heavy chain (SEQ ID NO: 16), and a Kozak sequence (SEQ ID NO: 26) was added to the 5′ end of the signal peptide sequence. Lastly, HindIII and NheI restriction sites were added to the 5′ and 3′ ends of the resulting sequence, respectively. For IMM5601's short chain, i.e., the anti-CD38 light chain, the same signal sequence as well as the Kozak sequence was added to the 5′ end of the anti-CD38 light chain coding sequence (SEQ ID NO: 18), and HindIII and XbaI restriction sites were added to the 5′ and 3′ ends of the resulting sequence, respectively. The sequences were synthesized by GenScript and cloned into pMac-H and pMac-L vectors, respectively.

For IMM5602's long chain, i.e., the anti-CD38 heavy chain, 57 nucleotides encoding the signal peptide of mouse IgG1 heavy chain (SEQ ID NO: 25) were added to the 5′ end of the coding sequence of the anti-CD38 heavy chain (SEQ ID NO: 20), and a Kozak sequence (SEQ ID NO: 26) was added to the 5′ end of the signal peptide sequence. Lastly, HindIII and NheI restriction sites were added to the 5′ and 3′ ends of the resulting sequence, respectively. For IMM5602's short chain, i.e., the SIRPαD1-linker-anti-CD38 light chain, the same signal sequence as well as the Kozak sequence was added to the 5′ end of the SIRPαDl-linker-anti-CD38 light chain coding sequence (SEQ ID NO: 22), and HindIII and XbaI restriction sites were added to the 5′ and 3′ ends of the resulting sequence, respectively. The sequences were synthesized by GenScript and cloned into pMac-H and pMac-L vectors, respectively.

These recombinant fusion proteins of the disclosure were expressed using CHO—S cells. Briefly, CHO—S cells were seeded at a density of 1×10⁶ cells/ml in TransFx-CTMCHO Transient transfection Medium (Hyclone) containing 6 mM glutamine one day before transient transfection. The heavy/long chain and light/short chain expression vectors, at a mass ratio of 1:1 and a total DNA amount of 1 μg/ml, were added to OPTI-MEM medium (Gibco) whose volume was 1/20 of that of the TransFx-CTMCHO Transient transfection Medium as used. PEI (polyethylenimine, MW 40,000, polysciences) at 1 mg/ml was added to OPTI-MEM medium (Gibco) whose volume was 1/20 of that of the TransFx-CTMCHO Transient transfection Medium as used. The PEI dilution was slowly added to, mixed and incubated at room temperature for 20 min with the diluted DNAs, at a PEI:DNA mass ratio of 4:1. Then, the DNA/PEI mixture was added to the cell cultures, and the cells were incubated in a 37° C. and 5% CO₂ cell culture incubator with shaking at 110 rpm. Transfection enhancer (1 mM sodium butyrate, 0.25% V/V DMSO) was added two days later, and the temperature was decreased to 33° C. When the cell viability dropped to ˜50%, the cell culture supernatant was harvested by centrifugation at 3000 rpm for 5 min, and subjected to protein purification using Protein A chromatography.

### Example 2. Exemplary Recombinant Fusion Protein Bound to CD38⁺CD47⁺ Raji Cells

CD38⁺CD47⁺ Raji cells in 100 μl culture medium (PBS with 1% BSA, also referred to as 1% BSA-PBS) at a cell density of 1×10⁶/ml were incubated with 100 μl serially diluted IMM5601, IMM5602, IMM56 and IgG in 1% BSA-PBS, respectively, at 4° C. for 1 h. Cells were washed with cold 1% BSA-PBS twice, and then incubated with FITC-conjugated secondary antibody against human IgG-Fc (Cat #F9512, Sigma) for 45 min. Cells were washed twice and re-suspended in 200 μl 1% BSA-PBS. Then, the cells were subject to FACS analysis using a flow cytometer (Merck Millipore, Guava® easyCyte 5HT).

As shown in FIG. 2, IMM5601 and IMM5602 bound to Raji cells with EC₅₀s at 123.0 nM and 93.54 nM, respectively. Their binding activities were comparable to that of the monospecific CD38 binding antibody IMM56 (EC₅₀=83.43 nM).

### Example 3. Exemplary Recombinant Fusion Protein Bound to CD38⁺CD47⁺ U266 Cells

CD38⁺CD47⁺ U266 cells in 100 μl 1% BSA-PBS at a cell density of 1×10⁶/ml were incubated with 100 μl serially diluted IMM5601, IMM5602, IMM56 and IgG in 1% BSA-PBS, respectively, at 4° C. for 1 h. Cells were washed with cold 1% BSA-PBS twice, and then incubated with FITC-conjugated secondary antibody against human IgG-Fc (Cat #F9512, Sigma) for 45 min. Cells were washed twice and re-suspended in 200 μl PBS. Then, the cells were subject to FACS analysis using a flow cytometer (Merck Millipore, Guava® easyCyte 5HT).

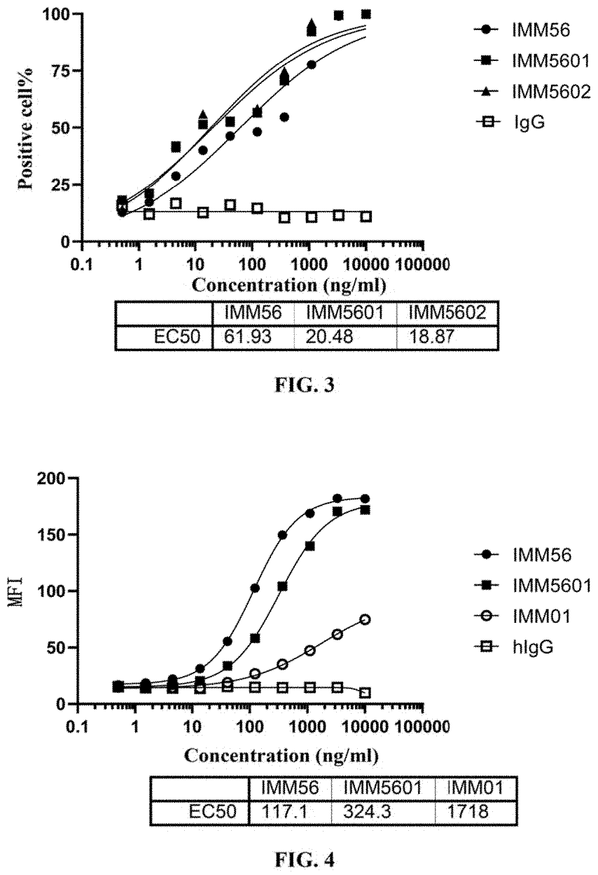

As shown in FIG. 3, IMM5601 and IMM5602 bound to U266 cells with EC₅₀s at 20.48 nM and 18.87 nM, respectively. Their binding activities were comparable to that of the monospecific CD38 binding antibody IMM56 (EC₅₀=61.93 nM).

### Example 4. Exemplary Recombinant Fusion Protein Bound to CD38⁺CD47⁺ NCI-H929 Cells

CD38⁺CD47⁺ NCI-H929 cells in 100 μl 1% BSA-PBS at a cell density of 1×10⁶/ml were incubated with 100 μl serially diluted IMM5601, IMM56, IMM01 and IgG in 1% BSA-PBS, respectively, at 4° C. for 1 h. Cells were washed with cold 1% BSA-PBS twice, and then incubated with FITC-conjugated secondary antibody against human IgG-Fc (Cat #F9512, Sigma) for 45 min. Cells were washed twice and re-suspended in 200 μl PBS. Then, the cells were subject to FACS analysis using a flow cytometer (Merck Millipore, Guava® easyCyte 5HT).

As shown in FIG. 4, IMM5601 bound to NCI-H929 cells with an EC₅₀ at 324.3 nM. IMM5601's binding activity was comparable to that of the monospecific CD38 binding antibody IMM56 (EC₅₀=117.1 nM), but much higher than that of the monospecific CD47 binding protein IMM01 (EC₅₀=1718 nM).

### Example 5. Exemplary Recombinant Fusion Protein Bound to CD38⁻CD47⁺ Jurkat Cells

CD38⁻CD47⁺ Jurkat cells in 100 μl 1% BSA-PBS at a cell density of 1×10⁶/ml were incubated with 100 μl serially diluted IMM5601, IMM5602, IMM56, IMM01 and IgG in 1% BSA-PBS, respectively, at 4° C. for 1 h. Cells were washed with cold 1% BSA-PBS twice, and then incubated with FITC-conjugated secondary antibody against human IgG-Fc (Cat #F9512, Sigma) for 45 min. Cells were washed twice and re-suspended in 200 μl PBS. Then, the cells were subject to FACS analysis using a flow cytometer (Merck Millipore, Guava® easyCyte 5HT).

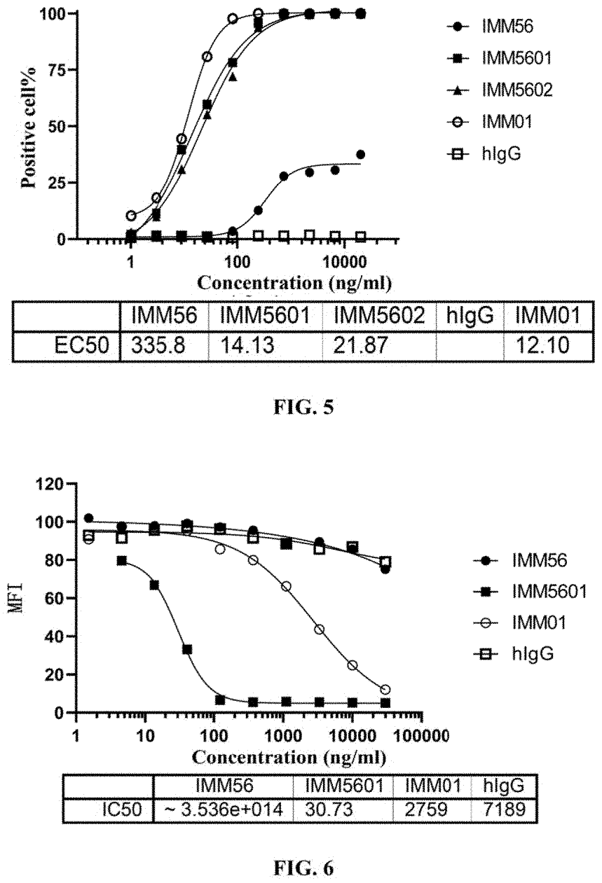

As shown in FIG. 5, IMM5601 and IMM5602 bound to Jurkat cells with EC₅₀s at 14.13 nM and 21.87 nM, respectively. Their binding activities were much higher than the monospecific CD38 binding antibody IMM56 (EC₅₀=335.8 nM), and a bit lower than that of the monospecific CD47 binding protein IMM01 (EC₅₀=12.1 nM).

### Example 5. Exemplary Recombinant Fusion Protein Inhibited CD47-SIRPα Interaction

SIRPα-mFc (wild-type human SIRPα with mouse IgG1 Fc, SEQ ID NO: 27) of 50 μl at 3 μg/ml was mixed with 50 μl serially diluted IMM5601, IMM56, IMM01, and hIgG-Fc (3-fold dilution, starting at 30 μg/ml), respectively. The resultant mixtures were added to the wells of a 96-well plate each containing 50 μl 1×10⁶/ml CD47⁺CD38⁺ NCI-H929 cells, and the plate was incubated at 4° C. for 45 min. The cells were washed with PBS and incubated with PE-conjugated secondary antibody against mouse IgG-Fc (Cat #405307, Biolegend) for 45 min. Cells were washed twice, re-suspended in 200 μl PBS, and subject to FACS analysis.

As shown in FIG. 6, IMM5601 blocked the binding of SIRPα-Fc to CD47⁺CD38⁺ cells at an IC₅₀ of 30.73 nM, with its inhibitory effect much higher than that of the monospecific CD47 binding protein IMM01 (IC₅₀=2759 nM).

### Example 7. Exemplary Recombinant Fusion Protein Induced High Antibody-Dependent Cell-Mediated Cytotoxicity (ADCC) Against CD38⁺CD47⁺ Cells

CFSE (Cat #21888-25 mg, Sigma) at 1 mM was 1:500 diluted and used to label CD38⁺CD47⁺ cells.

The CFSE-labeled Raji or U266 cells, as the target cells, of 50 μl at 6×10⁵/ml, were mixed at a 2:1 effector: target ratio with 100 μl 6×10⁵/ml NK92MI cells stably expressing FcγRIIIa, as the effector cells. The mixed cells were cultured for 4 hours at 37° C. under 5% CO₂ with 50 μl serially diluted IMM5601, IMM5602, IMM56 and IgG (3-fold dilution, starting at 1000 ng/ml), respectively. Then cell cultures were added with propidium iodide (PI) (Cat #P4170, Sigma) at a concentration of 5 μg/ml, and then subjected to FACS analysis for PI signals. Percent ADCC was calculated based on the following formula.

% Lysis=(% PI Positive Target Cells treated with IMM5601, IMM5602 or IMM56−% PI Positive Target Cells treated with negative control)/(100−% PI Positive Target Cells treated with negative control)*100

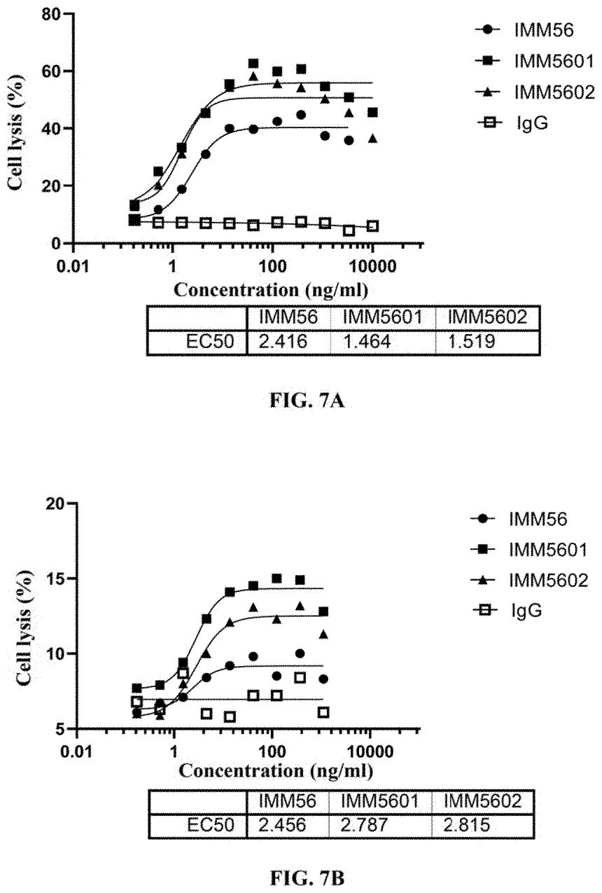

According to FIGS. 7A and 7B, IMM5601 and IMM5602 induced higher ADCC than IMM56 against Raji and U266 cells.

### Example 8. Exemplary Recombinant Fusion Protein Induced High Antibody-Dependent Cell-Mediated Cytotoxicity (ADCC) Against CD38⁺CD47⁺ NCI-H929 Cells

CFSE (Cat #21888-25 mg, Sigma) at 1 mM was 1:500 diluted and used to label NCI-H929 cells.

The CFSE-labeled NCI-H929 cells, as the target cells, of 50 μl at 6×10⁵/ml, were mixed at a 2:1 effector: target ratio with 100 μl 6×10⁵/ml NK92MI cells stably expressing FcγRIIIa, as the effector cells. The mixed cells were cultured for 4 hours at 37° C. under 5% CO₂ with 50 μl serially diluted IMM5601, IMM56, IMM01 and hIgG (3-fold dilution, starting at 1000 ng/ml), respectively. Then cell cultures were added with propidium iodide (PI) (Cat #P4170, Sigma) at a concentration of 5 μg/ml, and then subjected to FACS analysis for PI signals. Percent ADCC was calculated based on the following formula.

% Lysis=(% PI Positive Target Cells treated with IMM5601, IMM56 or IMM01−% PI Positive Target Cells treated with negative control)/(100−% PI Positive Target Cells treated with negative control)*100

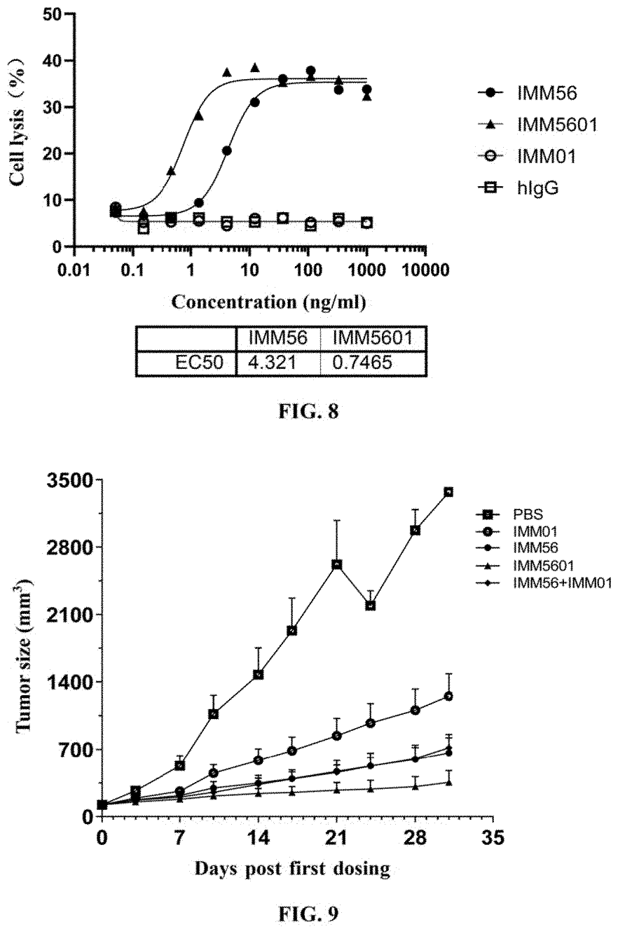

According to FIG. 8, IMM5601 induced higher ADCC against CD38⁺CD47⁺ NCI-H929 cells than IMM56 and IMM01.

### Example 9. Exemplary Recombinant Fusion Protein Showed Potent Anti-Tumor Activity

Thirty 6-8-week-old SCID mice were each subcutaneously injected at the right axilla with a mixture of 100 μl 5×10⁶ NCI-H929 cells in PBS and 100 μl Matrigel matrix. When tumor sizes reached 100-150 mm³, mice were randomly allocated into five groups with 6 mice per group, and this day was designated as Day 0. From that day on, mice were respectively given intraperitoneal injection of PBS, IMM01 (0.3 mg/kg), IMM56 (1.0 mg/kg), IMM5601 (1.2 mg/kg), and IMM01 (0.3 mg/kg)+IMM56 (1.0 mg/kg), for 4 weeks, twice per week. Administration was stopped at the end of week 4 and mice were observed for another 1 week. Mice tumor sizes and body weights were measured every 3-4 days.

The tumor volume (V) was calculated as (length×width²)/2. Tumor growth inhibition rate (TGI) was calculated by the formula: TGI (%)=(1−tumor volume change in administration group/tumor volume change in vehicle control group)×100%.

The test regime and results were summarized in Table 1.

TABLE 1
Anti-tumor effect of IMM5601 and other agents

| | | | Dose | | | P |
|---|---|---|---|---|---|---|
| Group | Drug | Animal# | (mg/kg) | Treatment | TGI | value |
| 1 | PBS | 6 | n/a | i.p.b.w. × 4 | | |
| 2 | IMM01 | 6 | 0.3 | i.p.b.w. × 4 | 72.12% | 0.012 |
| 3 | IMM56 | 6 | 1 | i.p.b.w. × 4 | 86.52% | 0.006 |
| 4 | IMM5601 | 6 | 1.2 | i.p.b.w. × 4 | 94.07% | 0.004 |
| 5 | IMM01 + | 6 | 0.3 + 1 | i.p.b.w. × 4 | 86.88% | 0.006 |
| | IMM56 | | | | | |

The results were shown in FIG. 9 and Table 1. On Day 21, the average tumor volume in Group 1 (PBS) was 2697.16 mm³ Compared to the vehicle control group, both IMM01 and IMM56 treatments significantly slowed tumor growth rate. These two groups had average tumor sizes at 840.23 mm³ (T/C=31.16%, TGI=72.12%, p=0.012) and 469.63 mm³ (T/C=17.40%, TGI=86.52%, p=0.006), respectively, on Day 21. The combined IMM56 and IMM01 administration also showed significant tumor suppression effects, with the mice having tumor sizes at 460.16 mm³ (T/C=17.06%, TGI=86.88%, p=0.006) on Day 21. Further, IMM5601 administration significantly inhibited tumor growth, with the tumor size being 274.79 mm³ on Day 21 (T/C=10.21%, TGI=94.07%, p=0.004), and two mice even achieved complete remission. It can be seen from FIG. 9 that IMM5601 exerted anti-tumor effect more efficiently than other agents.

In sum, IMM5601 functioned in an efficient and effective manner and produced greater in vivo anti-tumor efficacy, with the overall tumor inhibition effect being even better than the IMM56 and IMM01 combination treatment.

Description
Sequence/SEQ ID NO.

First extracellular Ig-like domain of SIRPα with mutation (SIRPαD1)
EEELQVIQPDKSVSVAAGESAILHCTVTSLIPVGPIQWFRGAGPARELIYNQKEGHFPRVTTVS
ESTKRENMDFSISISAITPADAGTYYCVKFRKGSPDTEFKSGAGTELSVRAKPSAPVVSGP
(SEQ ID NO: 1)
Heavy chain variable region of IMM56
QVQLVQSGAEVAKPGTSVKLSCKASGYTFTDYWMQWVKQRPGQGLEWIGTIYPGDGDTGY
AQKFQGKATLTADKSSKTVYMHLSSLASEDSAVYYCARGDYYGSNSLDYWGQGTSVTVSS
(SEQ ID NO: 2)
Light chain variable region of IMM56
DIVMTQSHLSMSTSLGDPVSITCKASQDVSTVVAWYQQKPGQSPRRLIYSASYRYIGVPDRFT
GSGAGTDFTFTISSVQAEDLAVYYCQQHYSPPYTFGGGTKLEIK (SEQ ID NO: 3)
HV-CDR-1 of IMM56
GYTFTDYW (SEQ ID NO: 4)
HV-CDR-2 of IMM56
IYPGDGDT (SEQ ID NO: 5)
HV-CDR-3 of IMM56
ARGDYYGSNSLDY (SEQ ID NO: 6)
LV-CDR-1 of IMM56
QDVSTV (SEQ ID NO: 7)
LV-CDR-2 of IMM56
SAS (SEQ ID NO: 8)
LV-CDR-3 of IMM56
QQHYSPPYT (SEQ ID NO: 9)
Heavy chain constant region
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREEQYNATYRVVSV
LTVLHQDWLNGKEYKCKVSNKALPAPIAATISKAKGQPREPQVYTLPPSREEMTKNQVSLTC
LVKGFYPSDIAVEWESNGQPENNYKTTPPVLDSDGSFFLYSKLTVDKSRWQQGNVFSCSVM
HEALHNHYTQKSLSLSPGK (SEQ ID NO: 10)
Light chain variable region
RTVAAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC (SEQ ID NO: 11)
Linker
GGGGSGGGGSGGGGS (SEQ ID NO: 12)
GGGGSGGGGS (SEQ ID NO: 13)
GGGGSGGGGSGGGGSGGGGS (SEQ ID NO: 14)
IMM5601’s long chain (SIRPαD1-linker-IMM56’s heavy chain)
EEELQVIQPDKSVSVAAGESAILHCTVTSLIPVGPIQWFRGAGPARELIYNQKEGHFPRVTTVS
ESTKRENMDFSISISAITPADAGTYYCVKFRKGSPDTEFKSGAGTELSVRAKPSAPVVSGPGG
GGSGGGGSGGGGSQVQLVQSGAEVAKPGTSVKLSCKASGYTFTDYWMQWVKQRPGQGLE
WIGTIYPGDGDTGYAQKFQGKATLTADKSSKTVYMHLSSLASEDSAVYYCARGDYYGSNSL
DYWGQGTSVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRVEPKSCDKTHTCPPC
PAPELLGGPSVFLFPPKPKDTLMISRTPEVTCVVVDVSHEDPEVKFNWYVDGVEVHNAKTKP
REEQYNATYRVVSVLTVLHQDWLNGKEYKCKVSNKALPAPIAATISKAKGQPREPQVYTLP
PSREEMTKNQVSLTCLVKGFYPSDIAVEWESNGQPENNYKTTPPVLDSDGSFFLYSKLTVDK
SRWQQGNVFSCSVMHEALHNHYTQKSLSLSPGK (SEQ ID NO: 15)
ATGGGATGGTCATGTATCATCCTTTTTCTGGTAGCAACTGCAACTGGAGTACATTCAGAG
GAGGAGCTGCAGGTGATTCAGCCTGACAAGTCCGTATCAGTTGCAGCTGGAGAGTCGGC
CATTCTGCACTGCACTGTGACCTCCCTGATCCCTGTGGGGCCCATCCAGTGGTTCAGAGG
AGCTGGACCAGCCCGGGAATTAATCTACAATCAAAAAGAAGGCCACTTCCCCCGGGTAA
CAACTGTTTCAGAGTCCACAAAGAGAGAAAACATGGACTTTTCCATCAGCATCAGTGCC
ATCACCCCAGCAGATGCCGGCACCTACTACTGTGTGAAGTTCCGGAAAGGGAGCCCTGA
CACGGAGTTTAAGTCTGGAGCAGGCACTGAGCTGTCTGTGCGTGCCAAACCCTCTGCCCC
CGTGGTATCGGGCCCTGGCGGCGGTGGGAGCGGCGGCGGTGGGAGCGGCGGCGGGGGC
TCGCAAGTGCAGCTGGTGCAGAGCGGCGCCGAGGTGGCCAAGCCTGGCACCTCCGTCAA
GCTGAGCTGCAAGGCCTCCGGCTACACCTTCACCGACTACTGGATGCAGTGGGTGAAGC
AGAGACCTGGCCAAGGCCTGGAGTGGATCGGCACCATCTACCCTGGCGACGGCGACACC
GGCTACGCTCAGAAGTTCCAAGGCAAGGCCACCCTGACCGCCGACAAGAGCAGCAAGAC
CGTGTACATGCACCTGAGCAGCCTGGCCTCCGAGGACAGCGCCGTGTACTACTGCGCTAG
AGGCGACTACTACGGCAGCAACAGCCTGGACTACTGGGGCCAAGGCACAAGCGTGACCG
TGAGCAGCGCTAGCACCAAGGGCCCATCGGTCTTCCCCCTGGCACCCTCCTCCAAGAGCA
CCTCTGGGGGCACAGCGGCCCTGGGCTGCCTGGTCAAGGACTACTTCCCCGAACCGGTG
ACGGTGTCGTGGAACTCAGGCGCCCTGACCAGCGGCGTGCACACCTTCCCGGCTGTCCTA
CAGTCCTCAGGACTCTACTCCCTCAGCAGCGTGGTGACCGTGCCCTCCAGCAGCTTGGGC
ACCCAGACCTACATCTGCAACGTGAATCACAAGCCCAGCAACACCAAGGTGGACAAGAG
AGTTGAGCCCAAATCTTGTGACAAAACTCACACATGCCCACCGTGCCCAGCACCTGAACT
CCTGGGGGGACCGTCAGTCTTCCTCTTCCCCCCAAAACCCAAGGACACCCTCATGATCTC
CCGGACCCCTGAGGTCACATGCGTGGTGGTGGACGTGAGCCACGAAGACCCTGAGGTCA
AGTTCAACTGGTATGTGGACGGCGTGGAGGTGCATAATGCCAAGACAAAGCCGCGGGAG
GAGCAGTACAACGCCACGTACCGTGTGGTCAGCGTCCTCACCGTCCTGCACCAAGACTG
GCTGAATGGCAAGGAGTACAAGTGCAAGGTCTCCAACAAAGCCCTCCCAGCCCCCATCG
CCGCAACCATCTCCAAAGCCAAAGGGCAGCCCCGAGAACCACAGGTGTACACCCTGCCC
CCATCCCGGGAGGAGATGACCAAGAACCAAGTCAGCCTGACCTGCCTGGTCAAAGGCTT
CTATCCCAGCGACATCGCCGTGGAGTGGGAGAGCAATGGGCAGCCGGAGAACAACTACA
AGACCACGCCTCCCGTGCTGGACTCCGACGGCTCCTTCTTCCTCTATTCCAAGCTCACCGT
GGACAAGAGCAGGTGGCAGCAGGGGAACGTCTTCTCATGCTCCGTGATGCATGAGGCTC
TGCACAACCACTACACGCAGAAGAGCCTCTCCCTGTCTCCGGGCAAATGA (SEQ ID NO:
16)
IMM5601’s short chain (IMM56’s light chain)
DIVMTQSHLSMSTSLGDPVSITCKASQDVSTVVAWYQQKPGQSPRRLIYSASYRYIGVPDRFT
GSGAGTDFTFTISSVQAEDLAVYYCQQHYSPPYTFGGGTKLEIKRTVAAPSVFIFPPSDEQLKS
GTASVVCLLNNFYPREAKVQWKVDNALQSGNSQESVTEQDSKDSTYSLSSTLTLSKADYEK
HKVYACEVTHQGLSSPVTKSFNRGEC (SEQ ID NO: 17)
ATGGGATGGTCATGTATCATCCTTTTTCTGGTAGCAACTGCAACTGGAGTACATTCAGAC
ATCGTGATGACACAGAGCCACCTGAGCATGAGCACAAGCCTGGGCGACCCTGTGAGCAT
CACCTGCAAGGCCTCCCAAGACGTGAGCACCGTGGTGGCCTGGTATCAGCAGAAGCCTG
GACAGAGCCCTAGAAGACTGATCTACAGCGCCTCCTACAGATACATCGGCGTGCCTGAC
AGATTCACCGGCAGCGGCGCCGGCACCGACTTCACCTTCACCATCAGCAGCGTGCAAGC
CGAGGACCTGGCCGTGTACTACTGTCAGCAGCACTACAGCCCTCCTTACACCTTCGGCGG
AGGCACCAAGCTGGAGATCAAGCGTGAGTTCTAGAGGATCCATCTGGGATAAGCATGCT
GTTTTCTGTCTGTCCCTAACATGCCCTGTGATTATCCGCAAACAACACACCCAAGGGCAG
AACTTTGTTACTTAAACACCATCCTGTTTGCTTCTTTCCTCAGGAACTGTGGCTGCACCAT
CTGTCTTCATCTTCCCGCCATCTGATGAGCAGTTGAAATCTGGAACTGCCTCTGTTGTGTG
CCTGCTGAATAACTTCTATCCCAGAGAGGCCAAAGTACAGTGGAAGGTGGATAACGCCC
TCCAATCGGGTAACTCCCAGGAGAGTGTCACAGAGCAGGACAGCAAGGACAGCACCTAC
AGCCTCAGCAGCACCCTGACGCTGAGCAAAGCAGACTACGAGAAACACAAAGTCTACGC
CTGCGAAGTCACCCATCAGGGCCTGAGCTCGCCCGTCACAAAGAGCTTCAACAGGGGAG
AGTGTTAG (SEQ ID NO: 18)
IMM5602’s long chain (IMM56’s heavy chain)
QVQLVQSGAEVAKPGTSVKLSCKASGYTFTDYWMQWVKQRPGQGLEWIGTIYPGDGDTGY
AQKFQGKATLTADKSSKTVYMHLSSLASEDSAVYYCARGDYYGSNSLDYWGQGTSVTVSS
ASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREEQYNATYRVVSV
LTVLHQDWLNGKEYKCKVSNKALPAPIAATISKAKGQPREPQVYTLPPSREEMTKNQVSLTC
LVKGFYPSDIAVEWESNGQPENNYKTTPPVLDSDGSFFLYSKLTVDKSRWQQGNVFSCSVM
HEALHNHYTQKSLSLSPGK (SEQ ID NO: 19)
ATGGGATGGTCATGTATCATCCTTTTTCTGGTAGCAACTGCAACTGGAGTACATTCACAA
GTGCAGCTGGTGCAGAGCGGCGCCGAGGTGGCCAAGCCTGGCACCTCCGTCAAGCTGAG
CTGCAAGGCCTCCGGCTACACCTTCACCGACTACTGGATGCAGTGGGTGAAGCAGAGAC
CTGGCCAAGGCCTGGAGTGGATCGGCACCATCTACCCTGGCGACGGCGACACCGGCTAC
GCTCAGAAGTTCCAAGGCAAGGCCACCCTGACCGCCGACAAGAGCAGCAAGACCGTGTA
CATGCACCTGAGCAGCCTGGCCTCCGAGGACAGCGCCGTGTACTACTGCGCTAGAGGCG
ACTACTACGGCAGCAACAGCCTGGACTACTGGGGCCAAGGCACAAGCGTGACCGTGAGC
AGCGCTAGCACCAAGGGCCCATCGGTCTTCCCCCTGGCACCCTCCTCCAAGAGCACCTCT
GGGGGCACAGCGGCCCTGGGCTGCCTGGTCAAGGACTACTTCCCCGAACCGGTGACGGT
GTCGTGGAACTCAGGCGCCCTGACCAGCGGCGTGCACACCTTCCCGGCTGTCCTACAGTC
CTCAGGACTCTACTCCCTCAGCAGCGTGGTGACCGTGCCCTCCAGCAGCTTGGGCACCCA
GACCTACATCTGCAACGTGAATCACAAGCCCAGCAACACCAAGGTGGACAAGAGAGTTG
AGCCCAAATCTTGTGACAAAACTCACACATGCCCACCGTGCCCAGCACCTGAACTCCTGG
GGGGACCGTCAGTCTTCCTCTTCCCCCCAAAACCCAAGGACACCCTCATGATCTCCCGGA
CCCCTGAGGTCACATGCGTGGTGGTGGACGTGAGCCACGAAGACCCTGAGGTCAAGTTC
AACTGGTATGTGGACGGCGTGGAGGTGCATAATGCCAAGACAAAGCCGCGGGAGGAGC
AGTACAACGCCACGTACCGTGTGGTCAGCGTCCTCACCGTCCTGCACCAAGACTGGCTGA
ATGGCAAGGAGTACAAGTGCAAGGTCTCCAACAAAGCCCTCCCAGCCCCCATCGCCGCA
ACCATCTCCAAAGCCAAAGGGCAGCCCCGAGAACCACAGGTGTACACCCTGCCCCCATC
CCGGGAGGAGATGACCAAGAACCAAGTCAGCCTGACCTGCCTGGTCAAAGGCTTCTATC
CCAGCGACATCGCCGTGGAGTGGGAGAGCAATGGGCAGCCGGAGAACAACTACAAGAC
CACGCCTCCCGTGCTGGACTCCGACGGCTCCTTCTTCCTCTATTCCAAGCTCACCGTGGAC
AAGAGCAGGTGGCAGCAGGGGAACGTCTTCTCATGCTCCGTGATGCATGAGGCTCTGCA
CAACCACTACACGCAGAAGAGCCTCTCCCTGTCTCCGGGCAAATGA (SEQ ID NO: 20)
IMM5602’s short chain (SIRPαD1-linker-IMM56’s light chain)
EEELQVIQPDKSVSVAAGESAILHCTVTSLIPVGPIQWFRGAGPARELIYNQKEGHFPRVTTVS
ESTKRENMDFSISISAITPADAGTYYCVKFRKGSPDTEFKSGAGTELSVRAKPSAPVVSGPGG
GGSGGGGSGGGGSDIVMTQSHLSMSTSLGDPVSITCKASQDVSTVVAWYQQKPGQSPRRLIY
SASYRYIGVPDRFTGSGAGTDFTFTISSVQAEDLAVYYCQQHYSPPYTFGGGTKLEIKRTVAA
PSVFIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC (SEQ ID NO: 21)
ATGGGATGGTCATGTATCATCCTTTTTCTGGTAGCAACTGCAACTGGAGTACATTCAGAG
GAGGAGCTGCAGGTGATTCAGCCTGACAAGTCCGTATCAGTTGCAGCTGGAGAGTCGGC
CATTCTGCACTGCACTGTGACCTCCCTGATCCCTGTGGGGCCCATCCAGTGGTTCAGAGG
AGCTGGACCAGCCCGGGAATTAATCTACAATCAAAAAGAAGGCCACTTCCCCCGGGTAA
CAACTGTTTCAGAGTCCACAAAGAGAGAAAACATGGACTTTTCCATCAGCATCAGTGCC
ATCACCCCAGCAGATGCCGGCACCTACTACTGTGTGAAGTTCCGGAAAGGGAGCCCTGA
CACGGAGTTTAAGTCTGGAGCAGGCACTGAGCTGTCTGTGCGTGCCAAACCCTCTGCCCC
CGTGGTATCGGGCCCTGGCGGCGGTGGGAGCGGCGGCGGTGGGAGCGGCGGCGGGGGC
TCGGACATCGTGATGACACAGAGCCACCTGAGCATGAGCACAAGCCTGGGCGACCCTGT
GAGCATCACCTGCAAGGCCTCCCAAGACGTGAGCACCGTGGTGGCCTGGTATCAGCAGA
AGCCTGGACAGAGCCCTAGAAGACTGATCTACAGCGCCTCCTACAGATACATCGGCGTG
CCTGACAGATTCACCGGCAGCGGCGCCGGCACCGACTTCACCTTCACCATCAGCAGCGTG
CAAGCCGAGGACCTGGCCGTGTACTACTGTCAGCAGCACTACAGCCCTCCTTACACCTTC
GGCGGAGGCACCAAGCTGGAGATCAAGCGTGAGTTCTAGAGGATCCATCTGGGATAAGC
ATGCTGTTTTCTGTCTGTCCCTAACATGCCCTGTGATTATCCGCAAACAACACACCCAAG
GGCAGAACTTTGTTACTTAAACACCATCCTGTTTGCTTCTTTCCTCAGGAACTGTGGCTGC
ACCATCTGTCTTCATCTTCCCGCCATCTGATGAGCAGTTGAAATCTGGAACTGCCTCTGTT
GTGTGCCTGCTGAATAACTTCTATCCCAGAGAGGCCAAAGTACAGTGGAAGGTGGATAA
CGCCCTCCAATCGGGTAACTCCCAGGAGAGTGTCACAGAGCAGGACAGCAAGGACAGCA
CCTACAGCCTCAGCAGCACCCTGACGCTGAGCAAAGCAGACTACGAGAAACACAAAGTC
TACGCCTGCGAAGTCACCCATCAGGGCCTGAGCTCGCCCGTCACAAAGAGCTTCAACAG
GGGAGAGTGTTAG (SEQ ID NO: 22)
SIRPαD1 mutant-Fc (IMMO1)
EEELQVIQPDKSVSVAAGESAILHCTVTSLIPVGPIQWFRGAGPARELIYNQKEGHFPRVTTVS
ESTKRENMDFSISISAITPADAGTYYCVKFRKGSPDTEFKSGAGTELSVRAKPSAPVVSGPAAR
ATPQHEPKSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG (SEQ ID NO: 23)
GAGGAGGAGCTGCAGGTGATTCAGCCTGACAAGTCCGTATCAGTTGCAGCTGGAGAGTC
GGCCATTCTGCACTGCACTGTGACCTCCCTGATCCCTGTGGGGCCCATCCAGTGGTTCAG
AGGAGCTGGACCAGCCCGGGAATTAATCTACAATCAAAAAGAAGGCCACTTCCCCCGGG
TAACAACTGTTTCAGAGTCCACAAAGAGAGAAAACATGGACTTTTCCATCAGCATCAGT
GCCATCACCCCAGCAGATGCCGGCACCTACTACTGTGTGAAGTTCCGGAAAGGGAGCCC
TGACACGGAGTTTAAGTCTGGAGCAGGCACTGAGCTGTCTGTGCGTGCCAAACCCTCTGC
CCCCGTGGTATCGGGCCCTGCGGCGAGGGCCACACCTCAGCACGAGCCCAAATCTTGTG
ACAAAACTCACACATGCCCACCGTGCCCAGCACCTGAACTCCTGGGGGGACCGTCAGTC
TTCCTCTTCCCCCCAAAACCCAAGGACACCCTCATGATCTCCCGGACCCCTGAGGTCACA
TGCGTGGTGGTGGACGTGAGCCACGAAGACCCTGAGGTCAAGTTCAACTGGTACGTGGA
CGGCGTGGAGGTGCATAATGCCAAGACAAAGCCGCGGGAGGAGCAGTACAACAGCACG
TACCGTGTGGTCAGCGTCCTCACCGTCCTGCACCAGGACTGGCTGAATGGCAAGGAGTAC
AAGTGCAAGGTCTCCAACAAAGCCCTCCCAGCCCCCATCGAGAAAACCATCTCCAAAGC
CAAAGGGCAGCCCCGAGAACCACAGGTGTACACCCTGCCCCCATCCCGGGATGAGCTGA
CCAAGAACCAGGTCAGCCTGACCTGCCTGGTCAAAGGCTTCTATCCCAGCGACATCGCCG
TGGAGTGGGAGAGCAATGGGCAGCCGGAGAACAACTACAAGACCACGCCTCCCGTGCTG
GACTCCGACGGCTCCTTCTTCCTCTACAGCAAGCTCACCGTGGACAAGAGCAGGTGGCAG
CAGGGGAACGTCTTCTCATGCTCCGTGATGCATGAGGCTCTGCACAACCACTACACGCAG
AAGAGCCTCTCCCTGTCTCCGGGTTGA (SEQ ID NO: 24)
Murine IgG1 heavy chain signal peptide
ATGGGATGGTCATGTATCATCCTTTTTCTGGTAGCAACTGCAACTGGAGTACATTCA (SEQ
ID NO: 25)
Kozak
GCCGCCACC (SEQ ID NO: 26)
Human SIRPα-murine IgG1 Fc
MGWSCIILFLVATATGVHSSCAWSGVAGEEELQVIQPDKSVSVAAGESAILHCTVTSLIPVGPI
QWFRGAGPARELIYNQKEGHFPRVTTVSESTKRENMDFSISISNITPADAGTYYCVKFRKGSP
DTEFKSGAGTELSVRAKPSAPVVSGPAARATPQHTVSFTCESHGFSPRDITLKWFKNGNELSD
FQTNVDPVGESVSYSIHSTAKVVLTREDVHSQVICEVAHVTLQGDPLRGTANLSETIRVPPTL
EVTQQPVRAENQVNVTCQVRKFYPQRLQLTWLENGNVSRTETASTVTENKDGTYNWMSWL
LVNVSAHRDDVKLTCQVEHDGQPAVSKSHDLKVSAHPKEQGSNTAAENTGSNERNEFVPRD
CGCKPCICTVPEVSSVFIFPPKPKDVLTITLTPKVTCVVVDISKDDPEVQFSWFVDDVEVHTAQ
TQPREEQFNSTFRSVSELPIMHQDWLNGKEFKCRVNSAAFPAPIEKTISKTKGRPKAPQVYTIP
PPKEQMAKDKVSLTCMITDFFPEDITVEWQWNGQPAENYKNTQPIMNTNGSYFVYSKLNVQ
KSNWEAGNTFTCSVLHEGLHNHHTEKSLSHSPGK (SEQ ID NO: 27)

While the application has been described above in connection with one or more embodiments, it should be understood that the application is not limited to those embodiments, and the description is intended to cover all alternatives, modifications, and equivalents, as may be included within the spirit and scope of the appended claims. All referenced cited herein are further incorporated by reference in their entirety.

### REFERENCES

1. Chen, L., Diao, L., Yang, Y., Yi, X., Rodriguez, B. L., Li, Y., Villalobos, P. A., Cascone, T., Liu, X., Tan, L., et al. (2018). CD38-Mediated Immunosuppression as a Mechanism of Tumor Cell Escape from PD-1/PD-L1 Blockade. Cancer Discovery 8, 1156-1175.
2. Dwivedi, S., Rendón-Huerta, E. P., Ortiz-Navarrete, V., and Montaño, L. F. (2021). CD38 and Regulation of the Immune Response Cells in Cancer. Journal of Oncology 2021, 6630295.
3. Fife B T, Pauken K E. The role of the PD-1 pathway in autoimmunity and peripheral tolerance. Annals of the New York Academy of Sciences. 2011, 1217: 45-59
4. Francisco L M, Sage P T, Sharpe A H. The PD-1 pathway in tolerance and autoimmunity. Immunological Reviews. 2010, 236: 219-42
5. Gardai S J, McPhillips K A, Frasch S C, Janssen W J, Starefeldt A, Murphy-Ullrich J E, Bratton D L, Oldenborg P A, Michalak M, Henson P M. Cell-surface calreticulin initiates clearance of viable or apoptotic cells through trans-activation of LRP on the phagocyte. Cell. 2005; 123:321-334
6. Gennaro, ed., Remington: The Science and Practice of Pharmacy, 20th Ed., Lippincott Williams & Wilkins 2003
7. J. R. Robinson, ed., Sustained and Controlled Release Drug Delivery Systems, Marcel Dekker, Inc., New York, 1978
8. Krejcik, J., Casneuf, T., Nijhof, I. S., Verbist, B., Bald, J., Plesner, T., Syed, K., Liu, K., van de Donk, N. W., Weiss, B. M., et al. (2016). Daratumumab depletes CD38+ immune regulatory cells, promotes T-cell expansion, and skews T-cell repertoire in multiple myeloma. Blood 128, 384-394.
9. Lee W Y, Weber D A, Laur O, Severson E A, McCall I, Jen R P, Chin A C, Wu T, Gernert K M, Parkos C A. Novel Structural Determinants on SIRPα that Mediate Binding to CD47. J Immunol. 2007, 179:7741-7750
10. Obeid M, Panaretakis T, Joza N, Tufi R, Tesniere A, van Endert P, Zitvogel L, Kroemer G. Calreticulin exposure is required for the immunogenicity of gamma-irradiation and UVC lightinduced apoptosis. Cell Death Differ. 2007, 14:1848-1850
11. Orr A W, Pedraza C E, Pallero M A, Elzie C A, Goicoechea S, Strickland D K, Murphy-Ullrich J E. Low density lipoprotein receptor-related protein is a calreticulin coreceptor that signals focal adhesion disassembly. J Cell Biol. 2003, 161:1179-1189
12. Shields R L, Namenuk A K, Hong K, Meng Y G, Rae J, Briggs J, Xie D, Lai J, Stadlen A, Li B, Fox J A, Presta L G. High Resolution Mapping of the Binding Site on Human IgG1 for FcγRI, FcγRII, FcγRIII, and FcRn and Design of IgG1 Variants with Improved Binding to the FcgR. JBC. 2001, 276:6591-6604
13. Suzanne L. Topalian, F. Stephen Hodi, Julie R. Brahmer, Scott N. Gettinger, David C. Smith, David F. McDermott, John D. Powderly, Richard D. Carvajal, Jeffrey A. Sosman, Michael B. Atkins, Philip D. Leming, David R. Spigel, Scott J. Antonia, Leora Horn, Charles G. Drake, Drew M. Pardoll, Lieping Chen, William H Sharfman, Robert A. Anders, Janis M. Taube, Tracee L. McMiller, Haiying Xu, Alan J. Korman, Maria Jure-Kunkel, Shruti Agrawal, Daniel McDonald, Georgia D. Kollia, Ashok Gupta, Jon M. Wigginton, and Mario Sznol. Safety, Activity, and Immune Correlates of Anti-PD-1 Antibody in Cancer, N Engl J Med 2012; 366:2443-2454
14. Theocharides, A. P. A.; Jin, L. Q.; Cheng, P. Y.; Prasolava, T. K.; Malko, A. V.; Ho, J. M.; Poeppl, A. G.; Rooijen, N. van; Minden, M. D.; Danska, J. S.; Dick, J.; Wang, J. C. Y. J. Exp. Med. 2012, Vol. 209 No. 10 1883-1899
15. Thompson R H, Gillett M D, Cheville J C, Lohse C M, Dong H, Webster W S, Krejci K G, Lobo J R, Sengupta S, Chen L, Zincke H, Blute M L, Strome S E, Leibovich B C, Kwon E D. Costimulatory B7-H1 in renal cell carcinoma patients: Indicator of tumor aggressiveness and potential therapeutic target. PNAS. 2004, 101 (49): 17174-9
16. Tseng D, Volkmer J P, Willingham S B, Contreras-Trujillo H, Fathman J W, Fernhoff N B, Seita J, Inlay M A, Weiskopf K, Miyanishi M, Weissman I L. Anti-CD47 antibody-mediated phagocytosis of cancer by macrophages primes an effective antitumor T-cell response. PNAS. 2013, 110:11103-11108
17. Usmani, S. Z., Weiss, B. M., Plesner, T., Bahlis, N. J., Belch, A., Lonial, S., Lokhorst, H. M., Voorhees, P. M., Richardson, P. G., Chari, A., et al. (2016). Clinical efficacy of daratumumab monotherapy in patients with heavily pretreated relapsed or refractory multiple myeloma. Blood 128, 37-44.
18. Vlahopoulos, S A. Aberrant control of NF-κB in cancer permits transcriptional and phenotypic plasticity, to curtail dependence on host tissue: molecular mode. Cancer biology & medicine. 2017, 14: 254-270
19. Shim H. Bispecific Antibodies and Antibody-Drug Conjugates for Cancer Therapy: Technological Considerations. Biomolecules. 2020 Feb. 26; 10 (3):360
20. Wang S, Chen K, Lei Q, Ma P, Yuan A Q, Zhao Y, Jiang Y, Fang H, Xing S, Fang Y, Jiang N, Miao H, Zhang M, Sun S, Yu Z, Tao W, Zhu Q, Nie Y, Li N. The state of the art of bispecific antibodies for treating human malignancies. EMBO Mol Med. 2021 Aug. 24:e14291. doi: 10.15252/emmm 202114291
21. Wo, Y. J., Gan, A. S. P., Lim, X., Tay, I. S. Y., Lim, S., Lim, J. C. T., and Yeong, J. P. S. (2020). The Roles of CD38 and CD157 in the Solid Tumor Microenvironment and Cancer Immunotherapy. Cells 9, 26.
22. Ohta A, Sitkovsky M. (2001) Role of G-protein-coupled adenosine receptors in downregulation of inflammation and protection from tissue damage. Nature. 414(6866):916-20

1. A recombinant fusion protein, comprising an anti-CD38 antibody or an antibody fragment thereof, and a CD47 binding peptide,
wherein the anti-CD38 antibody or antibody fragment thereof comprises a heavy chain variable region, a heavy chain constant region, and a light chain variable region, wherein the heavy chain variable region comprises a heavy chain variable region CDR-1 (HV-CDR1), a HV-CDR2 and a HV-CDR3 having amino acid sequences set forth in SEQ ID NOs: 4, 5 and 6, respectively, the light chain variable region comprises a light chain variable region CDR-1 (LV-CDR1), a LV-CDR2 and a LV-CDR3 having amino acid sequences set forth in SEQ ID NOs: 7, 8 and 9, respectively, and the heavy chain constant region has FcR binding affinity and is linked to C-terminus of the heavy chain variable region,
wherein the CD47 binding peptide comprises a signal-regulatory protein (SIRP) extracellular domain having an amino acid sequence having at least 95% sequence identity to SEQ ID NO: 1,
wherein the CD47 binding peptide is linked to the anti-CD38 antibody or antibody fragment thereof.

2. The recombinant fusion protein of claim 1, wherein at least one paratope of the anti-CD38 antibody or antibody fragment thereof is linked to the CD47 binding peptide at N-terminus of the heavy chain variable region or the light chain variable region constituting the paratope.

3. The recombinant fusion protein of claim 2, wherein each paratope of the anti-CD38 antibody or antibody fragment thereof is linked to the CD47 binding peptide at N-terminus of the heavy chain variable region or the light chain variable region constituting the paratope.

4. The recombinant fusion protein of claim 3, wherein each paratope of the anti-CD38 antibody or antibody fragment thereof is linked to the CD47 binding peptide at N-terminus of the heavy chain variable region constituting the paratope.

5. The recombinant fusion protein of claim 3, wherein each paratope of the anti-CD38 antibody or antibody fragment thereof is linked to the CD47 binding peptide at N-terminus of the light chain variable region constituting the paratope.

6. The recombinant fusion protein of claim 1, wherein the heavy chain variable region and the light chain variable region comprise amino acid sequences of SEQ ID NOs: 2 and 3, respectively.

7. The recombinant fusion protein of claim 1, wherein the heavy chain constant region comprises the amino acid sequence of SEQ ID NO: 10.

8. The recombinant fusion protein of claim 1, further comprising a light chain constant region having the amino acid sequence of SEQ ID NO: 11, linked to the C-terminus of the light chain variable region.

9. The recombinant fusion protein of claim 3, wherein the anti-CD38 antibody or antibody fragment thereof is linked to the CD47 binding peptide via a linker.

10. The recombinant fusion protein of claim 9, wherein the linker is -(Gly-Gly-Gly-Gly-Ser)₃-(SEQ ID NO: 12), -(Gly-Gly-Gly-Gly-Ser)₂-(SEQ ID NO: 13), or -(Gly-Gly-Gly-Gly-Ser)₄-(SEQ ID NO: 14).

11. The recombinant fusion protein of claim 9, comprising
i) a CD47 binding peptide-linker-anti-CD38 heavy chain variable region-heavy chain constant region fragment having the amino acid sequence of SEQ ID NO: 15, and an anti-CD38 light chain variable region-light chain constant region fragment having the amino acid sequence of SEQ ID NO: 17;
ii) a CD47 binding peptide-linker-anti-CD38 heavy chain variable region-heavy chain constant region fragment having the amino acid sequence of SEQ ID NO: 15, and an anti-CD38 light chain variable region fragment having the amino acid sequence of SEQ ID NO: 3; or
iii) an anti-CD38 heavy chain variable region-heavy chain constant region fragment having the amino acid sequence of SEQ ID NO: 19, and a CD47 binding peptide-linker-anti-CD38 light chain variable region-light chain constant region fragment having the amino acid sequence of SEQ ID NO: 21.

12. A nucleic acid molecule encoding the recombinant fusion protein of claim 1.

13. An expression vector comprising the nucleic acid molecule of claim 12.

14. A host cell comprising the expression vector of claim 13.

15. A pharmaceutical composition, comprising the recombinant fusion protein of claim 1, and at least one pharmaceutically acceptable excipient.

16. The pharmaceutical composition of claim 15, further comprising at least one pharmaceutically acceptable adjuvant.

17. A method for treating a disease associated with over-expression of CD47 and/or CD38 in a subject in need thereof, comprising administering to the subject the pharmaceutical composition of claim 15.

18. The method of claim 17, wherein the disease is selected from the group consisting of acute myelocytic leukemia (AML), chronic myelocytic leukemia (CML), acute lymphoblastic leukemia (ALL), lymphoma, multiple myeloma (MM), bladder cancer, ovarian cancer, prostate cancer, lung cancer, colon cancer, breast cancer, pancreatic adenocarcinoma, melanoma, glioma, esophageal cancer, and plasma cell myeloma.
